(19) United States
(12) Patent Application Publication
(10) Pub. No.: US 2025/0281423 A1
(43) Pub. Date: Sep. 11, 2025

(54) VESICLES AND USES THEREOF

(71) Applicant: EXOCURE BIOSCIENCES, INC., San Diego, CA (US)

(72) Inventors: Jan Lötvall, Göteborg (SE); Kyong-su Park, Göteborg (SE); Markus Bergqvist, Göteborg (SE)

(21) Appl. No.: 18/549,094

(22) PCT Filed: Mar. 9, 2022
(86) PCT No.: PCT/US2022/019585

§ 371 Date: Sep. 5, 2023

Related U.S. Application Data
(60) Provisional application No. 63/159,290, filed on Mar. 10, 2021.

(51) Int. Cl. A61K 9/51; A61K 31/496; A61K 31/542; A61K 38/08; A61K 45/06

(52) U.S. Cl. CPC A61K 9/5176; A61K 9/5192; A61K 31/496; A61K 31/542; A61K 38/08; A61K 45/06

(57) ABSTRACT

The present disclosure provides synthetic eukaryotic vesicles (SyEVs) prepared from exosomes secreted by eukaryotic cells. The exosomes have been exposed to a mild alkaline pH to expel the cytoplasmic components present in the exosomes while retaining native structure of cell surface proteins, such as, integrins. The present disclosure provides extruded ghost nanovesicles (exgNVs) loaded with an anti-inflammatory agent. These exgNVs enhance the anti-inflammatory effect of the anti-inflammatory agent. Methods of making such SyEVs and exgNVs and therapeutic uses of such SyEVs and exgNVs are also provided. The SyEVs and exgNVs may be produced from a human cell line genetically modified to express lymphocyte function-associated antigen-1 (LFA-1), where LFA-1 is located on surface of the SyEVs and exgNVs.

### CROSS-REFERENCE TO RELATED APPLICATIONS

This application is a 371 of PCT Patent Application No. PCT/US2022/019585, filed on Mar. 9, 2022, which claims priority benefit to U.S. Provisional Application No. 63/159,290, filed Mar. 10, 2021 which is herein incorporated by reference in its entirety.

### INCORPORATION BY REFERENCE OF SEQUENCE LISTING PROVIDED AS A TEXT FILE

A Sequence Listing is provided herewith as a text file, “EXOC-006-SEQ_LIST_ST25.TXT”,” created on Apr. 2, 2024 and having a size of 3073 bytes. The contents of the text file are incorporated by reference herein in their entirety

### INTRODUCTION

Delivery of therapeutic agents to cells such as in vivo cells is an area of extensive research. Delivery of gene expression modulators is of special interest. Also of interest is delivery of anti-inflammatory molecules to prevent and/or reduce inflammation since unchecked inflammation can cause tissue damage and prolong recovery. In addition to inflammatory diseases, inflammation is associated with many other diseases and even with treatments offered for such diseases.

There is a need to enhance the effect of anti-inflammatory agents as well as to increase efficiency of delivery of therapeutic agents. In addition, there is a need to reduce inflammation caused by administration of a therapeutic agent. The present disclosure addresses these and other needs.

### SUMMARY

The present disclosure provides synthetic eukaryotic vesicles (SyEVs) generated from eukaryotic cells and comprising functional integrin on surface of the SyEVs and substantially devoid of cytoplasmic content of the eukaryotic cells.

The present disclosure also provides extruded ghost nanovesicles (exgNVs) comprising an anti-inflammatory agent, wherein the exgNVs have an anti-inflammatory property, enhance anti-inflammatory effect of the anti-inflammatory agent, and are produced from a human cell, and wherein the anti-inflammatory agent is present in a therapeutically effective amount.

Methods of making such vesicles and therapeutic uses of such vesicles are also provided. These SyEVs and exgNVs may be used in preventing or treating conditions that may benefit from administration of anti-inflammatory agents. Such conditions include conditions that involve inflammation.

SyEVs and exgNVs may be produced from a human cell line genetically modified to express lymphocyte function-associated antigen-1 (LFA-1), where LFA-1 is located on surface of the SyEVs and exgNVs. As compared to exgNVs, SyEVs have a higher level of LFA-1 expression, e.g., 2 times or more, 3 times or more, 4 times or more, or 5 times or more, such as, 2-5 times higher expression of LFA-1 than the expression of LFA-1 in exgNVs. Thus, the SyEVs may be used for delivering a cargo, e.g., a therapeutic agent, to cells that express ICAM-1.

SyEVs and exgNVs may be produced from a human cell line genetically modified to express Macrophage-1 antigen (Mac-1), where Mac-1 is located on surface of the SyEVs and exgNVs.

### BRIEF DESCRIPTION OF DRAWINGS

FIG. 1A depicts steps for generation of extruded gNVs (exgNVs) according to an embodiment of the present disclosure involving disruption of cell by serial extrusion.

FIG. 1B depicts steps for generation of exgNVs loaded with an anti-inflammatory agent according to an embodiment of the present disclosure.

FIGS. 2A-2D shows synergistic anti-inflammatory effect of loading of exgNVs with a small molecule anti-inflammatory agent as compared to the anti-inflammatory effect of exgNVs or the small molecule.

FIG. 3A shows synergistic anti-inflammatory effect of loading of exgNVs with a large molecule anti-inflammatory agent as compared to the anti-inflammatory effect of exgNVs or the large molecule.

FIG. 3B shows anti-inflammatory effect in vivo of exgNVs loaded with an anti-inflammatory agent as compared to exgNVs alone.

FIG. 4A demonstrates loading of a siRNA in exgNVs according to an embodiment of the present disclosure.

FIG. 4B demonstrates loading of Cas9 protein in exgNVs according to an embodiment of the present disclosure.

FIG. 5A provides a comparison of DNA content of gNVs prepared without extrusion and exgNVs.

FIG. 5B provides a comparison of protein profile of gNVs prepared without extrusion and exgNVs.

FIG. 6A compares effect of gNVs prepared without extrusion and exgNVs on level of outer membrane vesicle (OMV)-induced TNFα.

FIG. 6B compares effect of gNVs prepared without extrusion and exgNVs on level of OMV-induced IL-6.

FIG. 7A shows Nano-FCM data on NVs prepared from 293f cells expressing LFA-1 that indicates that the NVs maintained expression.

FIG. 7B shows Western blot data on NVs prepared from 293f cells expressing LFA-1 that indicates that the NVs maintained expression.

FIG. 7C compares the binding of LFA-1 expressing cells to wells coated with ICAM-1 and non-coated wells.

FIG. 8A shows the flow cytometry analysis of the cells transfected with CD18.

FIG. 8B shows the flow cytometry analysis of the cells transfected with CD11a.

FIG. 9A depicts steps for generation of synthetic eukaryotic vesicles (SyEV) encapsulated with Cas9 proteins.

FIG. 9B shows Western blot analysis of SyEV loaded with Cas9 with anti-Cas9 antibody using different combinations of incubation conditions.

FIG. 10A depicts steps for generation of SyEV loaded with CRISPR complex and functional assay.

FIG. 10B shows GFP signals of GFP-overexpressing cell lines treated with 10¹⁰ for SyEV^{CRISPR} or lipofectamine for 48 hours. The data were analyzed by flow cytometry and shown as the relative percentage of GFP signals of PBS group.

FIG. 11A shows Western blot data on EV prepared from 293f cells expressing LFA-1 that indicates maintained expression of LFA-1 in the EV.

FIG. 11B shows Nano-FCM data on EV isolated from 293f cells expressing LFA-1 that indicates maintained expression of LFA-1 in the EV.

FIG. 11C shows Nano-FCM analysis of exosomes (EVs) isolated from 293f cells genetically modified to overexpress LFA-1, NVs prepared by serial extrusion of 293f cells genetically modified to overexpress LFA-1, EVs isolated from 293f cells genetically modified to overexpress CD18, and NVs prepared by serial extrusion of 293f cells genetically modified to overexpress CD18.

FIG. 12 depicts a binding assay with ICAM-1 expressing cells and EV isolated from different kinds of HEK293f clones used as coating. EV used were from a LFA-1 clone, CD18 clone and 293f-WT. Also, neutralized LFA-1 EV group was included.

FIG. 13A depicts TNF-α treated ICAM-1 expressing cells incubated with DiO stained EV isolated from different HEK293f clones. EV used were isolated from an LFA-1 clone, CD18 clone and 293f-WT. Included are cells incubated with neutralized LFA-1 EV.

FIG. 13B depicts TNF-α treated and non-treated ICAM-1 expressing cells incubated with DiO stained LFA-1 EV. Included is cells incubated with neutralized LFA-1 EV.

FIG. 14A depicts effect of different pH treatments on the binding of LFA-1 SyEV to ICAM-1 expressing cells by using reverse binding assays.

FIG. 14B shows schematic diagram of a method of preparing LFA-1 SyEV loaded with anti-Myd88 peptides.

FIG. 15 shows uptake analysis of peptide-encapsulated LFA-1 SyEV in the activated endothelial cells overexpressing ICAM-1.

FIG. 16 shows uptake analysis of peptide-encapsulated LFA-1 SyEV in the activated endothelial cells overexpressing ICAM-1, which is especially compared to peptide-encapsulated wild-type SyEV.

FIG. 17 shows Nano-FCM data on EV isolated from 293f cells expressing Mac-1 that indicates maintained expression of Mac-1 in the EV.

FIG. 18 depicts a binding assay with ICAM-1 expressing cells and EV isolated from different kinds of HEK293F clones used as coating. EV used were from a Mac-1 clone, LFA-1 clone, CD18 clone and 293F-WT.

### DEFINITIONS

The term “vesicle” as used herein refers to a spherical structure which contains an interior volume that is separated from the outside environment by a lipid bilayer membrane. A vesicle can be secreted from cells or can be artificially synthesized from a cell, such as, a eukaryotic cell. A vesicle is generally smaller than the cell from which it is derived.

The term “revesiculation” and grammatical equivalents thereof, as used herein refers to a process of opening a vesicle, e.g., a cell-derived vesicle, such that the interior contents of the vesicle are released, followed by isolation of the open lipid bilayer membrane, and closing of the open lipid bilayer membrane to reform vesicles. Such vesicles are referred to as ghost vesicles.

The term “non-revesiculated” and grammatical equivalents thereof, as used herein refers to a vesicle, e.g., a cell-derived vesicle that is not a ghost vesicle, i.e., has not been subjected to the process of opening the vesicle such that the interior contents of the vesicle are released, followed by isolation of the open lipid bilayer membrane, and closing of the open lipid bilayer membrane to reform vesicles. Thus, a non-revesiculated vesicle encloses significantly more of the interior contents from the cell from which it is derived as compared to a ghost vesicle prepared from the same type of cell.

The term “deficient” as used in the context of a component present in the ghost nanovesicles (gNVs) derived from a cell as disclosed herein means having at least 50% less of the component, for example, 60%, 70%, 80%, 90%, or 99%, as compared to amount of the component present in non-ghost nanovesicles produced from the same cell.

Vesicles that have not been prepared by opening and closing of the vesicles are referred to as nanovesicles (NVs). Vesicles prepared by opening and closing of vesicles are referred to herein as ghost NVs (gNVs). Vesicles prepared from serial extrusion of a cell, followed by opening and closing of vesicles, are referred to herein as extruded ghost NVs (exgNVs).

The term “enriched” as used in the context of a protein (e.g., a membrane protein) present in the gNVs (e.g., exgNVs) derived from a cell as disclosed herein means that the component makes up a bigger fraction of the total amount of protein in the gNVs as compared to the fraction of the same protein in NVs produced from the same cell type. For example, the enriched protein may represent at least 25% or more of the total proteins in the gNVs while the same protein may represent at most 20% of the total proteins in the NVs. An enriched component may be present in the gNVs at a higher concentration by total weight, e.g., at least a three-fold greater concentration by total weight, e.g., at least 5-fold greater concentration, at least 10-fold greater concentration, at least 30-fold greater concentration, at least 50-fold greater concentration, or at least 100-fold greater concentration than the concentration of that component by total weight in NVs generated from the same cell type from which the gNVs were derived. Thus, if the concentration of a particular component is 1 microgram per gram of total cell preparation (or of total cell protein), an enriched preparation would contain greater, e.g., at least 3 micrograms of the component per gram of total cell preparation (or of total cell protein).

As used herein, the term “extracellular vesicle” means a vesicle released by a eukaryotic, e.g., a mammalian cell. Examples of “extracellular vesicles” include exosomes, ectosomes, microvesicles, prostasomes, oncosomes, and apoptotic bodies. As used herein, the term “tumor vesicle” refers to an extracellular vesicle present in a tumor tissue, e.g., released by a tumor cell. A tumor vesicle may be opened and closed to produce a gTV such as ghost tumor micro or nanovesicles (gTMVs or gTNVs). In certain embodiments, the gNVs are not generated from tumor vesicles.

The term “inflammatory response” as used herein refers to secretion of proinflammatory cytokines, activation of toll-like receptors (TLR) and/or systemic inflammation. Examples of proinflammatory cytokines include -6 IL-2, IL-4, IL-6, IL-12, IL-12p70, IL-17, tumor necrosis factor alpha (TNF-α) and interferon gamma (IFN-γ).

The term “outer membrane vesicle(s)” or “OMV(s)” as used herein refers to vesicles that include an outer membrane enclosing periplasmic contents, cytoplasmic contents and inner membrane components of a bacterium, e.g., a gram-negative bacterium. OMVs include blebs produced by budding of the outer membrane of organisms, such as, gram-negative bacteria. Such OMVs can also be referred to as native OMVs. OMVs can also be produced by disrupting (e.g., by extrusion, sonication, detergents, or osmotic shock) a gram-negative bacterium in a hydrophilic solution thereby forcing the cell to form vesicles.

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

The terms “subject” and “patient” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treatment,” “treat,” or “treating,” as used herein cover any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

The term “therapeutic effect” refers to some extent of relief of one or more of the symptoms of a disorder (e.g., infection, a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the vesicles of the present disclosure employed in a pharmaceutical composition at levels lower than that required in order to achieve the desired therapeutic effect and gradually increase the dosage until the desired effect is achieved.

The phrase “conservative amino acid substitution” refers to substitution of amino acid residues within the following groups: 1) L, I, M, V, F; 2) R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a vesicle” includes a plurality of such vesicles and reference to “the vesicle” includes reference to one or more vesicles and equivalents thereof known to those skilled in the art and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication. Further, the dates of publication provided may be different from the actual publication dates which may need to be independently confirmed.

### DETAILED DESCRIPTION

The present disclosure provides ghost nanovesicles (gNVs), e.g., extruded ghost NVs (exgNVs), comprising an anti-inflammatory agent, wherein the gNVs have an anti-inflammatory property, enhance anti-inflammatory effect of the anti-inflammatory agent, and are produced from a human cell, and wherein the anti-inflammatory agent is present in a therapeutically effective amount. Methods of making such vesicles and therapeutic uses of such vesicles are also provided. These gNVs may be used in preventing or treating conditions that may benefit from administration of anti-inflammatory agents. Such conditions include conditions that involve inflammation.

In certain embodiments, the gNVs may be extruded gNVs, where the initial NVs are prepared by serial extrusion of human cells. The initial NVs are subjected to an alkaline pH which results in opening of the NVs and release of intracellular content of the initial NVs. The vesicles formed after release of intracellular contents of NVs prepared by extrusion of human cells are referred to as exgNVs.

In certain embodiments, the gNVs may be synthetic gNVs, where the initial NVs are not prepared by serial extrusion of human cells. Rather, the initial NVs are exosomes naturally produced by the human cells. These exosomes are subjected to an alkaline pH which results in opening of the exosomes and release of intracellular content of the exosomes. The vesicles formed after release of intracellular contents of the exosomes are referred to as synthetic eukaryotic vesicles SyEVs. As compared to exgNVs, the SyEVs have a higher level of cell surface proteins, such as, LFA-1 and/or Mac-1. SyEVs may be used in methods where the target cell to which the gNVs are delivering their cargo bind to cell surface proteins, such as, LFA-1 and/or Mac-1.

Also disclosed are vesicles, such as, nanovesicles (NVs) (e.g., naturally produced NVs (also referred to as exosomes), gNVs, exgNVs, SyEVs, etc.) produced from a human cell line genetically modified to express an integrin, e.g., lymphocyte function-associated antigen-1 (LFA-1) or Mac-1, where LFA-1 or Mac-1 is located on surface of the vesicles. These NVs, when loaded with an agent, may be used to deliver the agent to a cell expressing a receptor for LFA-1 or Mac-1. In certain embodiments, SyEVs may be used to deliver the agent to a cell expressing a receptor for LFA-1 or Mac-1 since the SyEVs have a higher level of cell surface proteins, such as, LFA-1 and/or Mac-1, as compared to exgNVs.

The NVs provided herein can also be used to package and deliver agents, e.g., a Cas protein, or a Cas protein and a guide RNA to a cell. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, the vesicles may be produced from a human stem cell line, e.g., mesenchymal stem cell (MSC). Data provided herein demonstrates that SyEVs produced from MSCs are surprisingly more efficient in delivering a Cas protein guide RNA to a target cell than SyEVs produced from HEK293 cells.

Extruded Ghost Nanovesicles Loaded with an Anti-Inflammatory Agent

Extruded ghost nanovesicles (exgNVs) comprising an anti-inflammatory agent are disclosed. The exgNVs have an anti-inflammatory property, enhance anti-inflammatory effect of the anti-inflammatory agent, and are produced from a human cell. The anti-inflammatory agent is present in the exgNVs in a therapeutically effective amount.

These exgNVs are deficient in cytoplasmic proteins and nucleic acids and reduce the levels of at least one proinflammatory cytokine when administered to a subject in need thereof are disclosed. These exgNVs provide a synergistic anti-inflammatory effect when loaded with an anti-inflammatory agent. As used herein, the term “synergistic anti-inflammatory effect” as used in the context of exgNVs comprising an anti-inflammatory agent refers to an anti-inflammatory effect that is greater than the expected sum of each of the anti-inflammatory effect of exgNVs alone and that of the anti-inflammatory agent alone.

In certain embodiments, the human cell may be an autologous human cell. The autologous human cell may be derived from a tissue or organ of the subject. In certain embodiments, the human cell may be a heterologous human cell. In certain embodiments, the heterologous human cell may be derived from a tissue or organ of a donor, or from a cell line. The tissue or organ from which the human cell is derived may be bone marrow, blood, blood product, adipose tissue, cord blood, fallopian tube, liver, fetal liver or fetal lungs, etc. In certain embodiments, the human cell may be monocytes, macrophages, or dendritic cells. In certain embodiments, the human cell may not be monocytes, macrophages, or dendritic cells. In certain embodiments, the human cell may not be a cancer cell or a cell derived from a tumor.

In certain embodiments, the human cell may be a stem cell. In certain embodiments, the human cell may be a cell line, such as, a human embryonic stem cell line, an induced pluripotent human stem cell or any other human cell line. In certain embodiments, the human stem cell may be an embryonic stem cell or a somatic stem cell such as those found in children and adults. In certain embodiments, the human cell may be a hematopoietic stem cell, a mammary stem cell, an intestinal stem cell, an endothelial stem cell, a neural stem cell, an olfactory stem cell, a neural crest stem cell, or a testicular stem cell. In particular embodiments, the human cell may be a mesenchymal stem cell. In other embodiments, the human cell may be a cell line that has been genetically modified, to change the content and cargo of the exgNV.

In certain embodiments, the human cell may be genetically modified. The human cell may be genetically modified to express a therapeutic agent, such as, a membrane protein or a lipid that is localized to the plasma membrane. In certain embodiments, the human cell may be a cell that naturally produces or is genetically modified to produce one or more therapeutic agents, such as, adhesion molecules, such as, integrins; protein kinases, e.g., tyrosine kinases, serine/threonine kinases; transcription factors; ion channels, e.g., calcium channels, potassium channels, sodium channels; growth factors; interleukins; neurotrophic factors, etc.

In certain embodiments, the human cell may be a cell that naturally produces or is genetically modified to produce one or more therapeutic agents, such as, trophic factors, e.g., CDNF, GDNF, neurturin, IGF1, VEGF, HGF; chaperones, e.g., HSP104, HSP70; ephA4; ephA4 ligands; Poly(A) Binding Protein Nuclear 1 (PABPN1); matrin ubiquilin 2; Zinc finger protein 106 (ZFP106); IRE1a kinase/Rnase; ubiquilins; TANK Binding Kinase 1 (TBK1); MuSK agonist antibodies; Ankyrin Repeat And KH Domain Containing 1 (ANKHD1); affitins; Glycerophosphodiester phosphodiesterase 2 (GDE2); MMIF; SRSF1 nuclear transport; anti-mir155; miRNA 125b; miRNA 31; miRNA-206; miRNA 133b; TREM2 activating antibodies; SARM1 inhibitor; macrophage migration inhibitory factor (MIF); dominant negative NFkB; Muscle-Specific Kinase; siRNA targeting tristetraprolin; PPARγ CoActivator 1alpha; Ret Receptor; notch intracellular domain; TGFβ; INFγ, etc.

The exgNVs provided herein retain the membrane proteins which membrane proteins are in substantially native conformation. For example, the exgNVs are not exposed to denaturants used as vesiculation agents during generation of the gNVs. For example, the method for making the exgNVs does not involve a step of exposing the human cell to a vesiculation agent to form vesicles. In other words, the exgNVs are not exposed to vesiculation agents, such as, sulfhydryl blocking agent during or after generation of the exgNVs such that the exgNVs retain the membrane proteins in their native conformation. The exgNVs are not exposed to during or after formation to vesiculation agents such as formaldehyde and dithiothreitol. Sulfhydryl blocking agents include formaldehyde, pyruvic aldehyde, acetaldehyde, glyoxal, glutar aldehyde, acrolein, methacrolein, pyridoxal, N-ethyl malemide (NEM), malemide, chloromercuribenzoate, iodoacetate, potassium arsenite, sodium selenite, thimerosal (merthiolate), benzoyl peroxide, cadmium chloride, hydrogen peroxide, iodosobenzoic acid, meralluride sodium, (mercuhydrin), mercuric chloride, mercurous chloride, chlormerodrin (neohydrin), phenylhydrazine, potassium tellurite, sodium malonate, p-arsenosobenzoic acid, 5,5′-diamino-2, 2′-dimethyl arsenobenzene, N,N′-dimethylene sulfonate disodium salt, iodoacetamide, oxophenarsine (mapharsen), auric chloride, p-chloromercuribenzoic acid, p-chloromercuriphenylsulfonic acid, cupric chloride, iodine merbromin (mercuro chrome) porphyrindine, potassium permanganate, mersalyl (salyrgan), silver nitrate, strong silver protein (protargol), uranyl acetate, etc. Other examples of vesiculation agents include cell toxins such as cytochalasin B or melittin.

As used herein, the phrase “not exposed to” in the context of a vesiculation agent means that the exgNVs are not exposed to a substantial amount of the vesiculation agent which amount is sufficient to cause generation of vesicles. In other words, the exgNVs may be exposed to during or after generation to trace amounts of a vesiculation agent which does not cause denaturation of membrane proteins and does not cause formation of vesicles.

In certain embodiments, the exgNVs provided herein may be distinguished from gNVs generated by using a vesiculation agent by assaying the vesicles. Assays such as immunoassay or functional assays may be used. In certain embodiments, an antibody that binds to a membrane protein in native conformation but does not bind to the protein when it is denatured may be used in an immunoassay to distinguish the exgNVs from gNVs made using a vesiculation agent. A functional assay may involve assaying the exgNVs for activity of a membrane protein such as binding to a ligand, uptake of a ligand, ability to deliver or pump out a drug or take up a molecule, and the like.

The exgNVs may be roughly spherical in shape and may have a diameter smaller than the cells from which the exgNVs are produced. In certain embodiments, exgNVs may be relatively large exgNVs that may range in diameter from 100 nm-900 nm, e.g., 100 nm-800 nm, 100 nm-700 nm, 100 nm-600 nm, 100 nm-500 nm, 100 nm-400 nm, 100 nm-300 nm, or 100 nm-200 nm. In certain embodiments, exgNVs may be relatively small gNVs that may range in diameter from 10 nm-100 nm, e.g., 20 nm-100 nm, 30 nm-100 nm, or 40 nm-100 nm. In certain embodiments, a preparation of exgNVs, such as a composition of exgNVs may include large and small exgNVs.

The SyEVs may be roughly spherical in shape and may have a diameter smaller than the cells from which the SyEVs are produced. In certain embodiments, SyEVs may be relatively large SyEVs that may range in diameter from 100 nm-900 nm, e.g., 100 nm-800 nm, 100 nm-700 nm, 100 nm-600 nm, 100 nm-500 nm, 100 nm-400 nm, 100 nm-300 nm, or 100 nm-200 nm. In certain embodiments, SyEVs may be relatively small SyEVs that may range in diameter from 10 nm-100 nm, e.g., 20 nm-100 nm, 30 nm-100 nm, or 40 nm-100 nm. In certain embodiments, a preparation of SyEVs, such as a composition of SyEVs may include large and small SyEVs.

In certain aspects, a exgNV may be formed by disrupting the mammalian cell to generate vesicles; separating the vesicles using a density gradient and isolating nanovesicles; exposing the isolated nanovesicles to an alkaline pH to open the nanovesicles thereby generating plasma membrane sheets; purifying the plasma membrane sheets; and applying energy to the purified plasma membrane sheets sufficient to convert the plasma membrane sheets into exgNVs.

In certain embodiments, exgNVs may be formed by serially extruding the human cell to generate vesicles; separating the vesicles using a density gradient and isolating nanovesicles; exposing the isolated nanovesicles to an alkaline pH to open the nanovesicles thereby generating plasma membrane sheets; purifying the plasma membrane sheets; and applying energy to the purified plasma membrane sheets sufficient to convert the plasma membrane sheets into exgNVs. NVs formed by methods disclosed herein includes cytoplasmic components, such as, organelles, cytoplasmic proteins, nucleus, nucleic acids (e.g., RNA, such as, mRNA, miRNA, and the like). exgNV are deficient in such components, i.e., have at least 50% less of the component, for example, 60%, 70%, 80%, 90%, or 99% less, as compared to amount of the component present in the NVs. gNVs produced by a disrupting step that involves serial extrusion are referred to herein as extruded gNVs (exgNVs).

In certain embodiments, SyEVs may be formed by isolating exosomes secreted by eukaryotic cells. Isolating may involve separating the exosomes from cells and/or cell debris using a density gradient and isolating exosomes; exposing the isolated exosomes to a mild alkaline pH to open the nanovesicles thereby generating plasma membrane sheets; purifying the plasma membrane sheets; and applying energy to the purified plasma membrane sheets sufficient to convert the plasma membrane sheets into SyEVs. Exosomes include cytoplasmic components, such as, organelles, cytoplasmic proteins, nucleus, nucleic acids (e.g., RNA, such as, mRNA, miRNA, and the like) as well as membrane proteins. SyEVs are deficient in cytoplasmic components, i.e., have at least 50% less of the cytoplasmic component, for example, 60%, 70%, 80%, 90%, or 99% less, as compared to amount of the component present in the exosomes from which the SyEVs are derived. Use of mild alkaline pH (e.g., an alkaline pH of less than 10, such as, pH7.5-pH9.5, pH8-pH9.5, or pH8.5-pH9.5) results in retention of functional membrane proteins on the surface of the SyEVs such that the SyEVs comprises a higher amount of functional membrane proteins on the surface as compared to the amount of functional membrane proteins on the surface of vesicles prepared from exosomes by exposing the exosomes to a high alkaline pH, such as, pH 10 or higher (e.g., pH10 or pH11). For example, the SyEVs of the present disclosure include 2 times or more, 3 times or more, 4 times or more, or 5 times or more functional membrane proteins on the surface as compared to the amount of functional membrane proteins on the surface vesicles prepared from exosomes by exposing the exosomes to a high alkaline pH. The membrane proteins may comprise LFA-1 and/or Mac-1. In certain embodiments, the SyEVs are generated from human cells genetically modified to overexpress LFA-1.

In certain embodiments, the exgNVs or the SyEVs are made by adding an anti-inflammatory agent to a composition comprising the purified membrane sheets and applying energy to the composition sufficient to convert the plasma membrane sheets into exgNVs or the SyEVs comprising the anti-inflammatory agent. In certain embodiments, the anti-inflammatory agent is present in the exgNVs or the SyEVs in a therapeutically effective amount. In other words, the average amount of anti-inflammatory agent incorporated into the exgNVs or the SyEVs is a therapeutically effective amount. In certain embodiments, the amount of the anti-inflammatory agent that is therapeutically effective when the agent is present in the exgNVs or the SyEVs is significantly less than the amount needed to achieve the same therapeutic effect when the agent is administered by itself.

The exgNVs or the SyEVs may be loaded with an anti-inflammatory agent that is a small molecule or a large molecule. The large molecule may be a peptide, protein, aptamer, or a nucleic acid. The exgNVs or the SyEVs may be loaded with an anti-inflammatory agent that is an inhibitor of Myeloid Differentiation Primary Response 88 (Myd88), cyclosporin A (CsA), an inhibitor of a non-receptor tyrosine kinase (non-RTK), an inhibitor of Nuclear factor kappa B (NFkB) p50, AKT, extracellular-signal-regulated kinase (ERK), I kappa B kinase gamma (IKKg), Toll Like Receptor 4 (TLR4), Toll Like Receptor 2 (TLR2), Signal transducer and activator of transcription 3 (STAT3), or interleukin-1 receptor-associated kinase 4 (IRAK), or an activator of glucocorticoid receptor.

In certain embodiments, the anti-inflammatory agent is a small molecule. The small molecule may be cyclosporin A (CsA). In another embodiment, the small molecule may be an inhibitor of Myd88. In some embodiments, the small molecule inhibitor of Myd88 may be T6167923 or ST2825. In certain embodiments, the small molecule is an inhibitor of a non-receptor tyrosine kinase. The non-receptor tyrosine kinase may be a member of Abelson (Abl), Feline Sarcoma (FES), JAK, Activated Cdc42 kinases (ACK), Spleen tyrosine kinase (SYK), TEC, Focal adhesion kinase (FAK), Src, or C-terminal Src kinases (CSK) family of kinases. In certain embodiments, the non-receptor tyrosine kinase may be Abl1 or Abl2. In certain embodiments, the non-receptor tyrosine kinase may be JAK1, JAK2, JAK3, or Tyk2. In certain embodiments, the non-receptor tyrosine kinase may be Ack1/Tnk2, Ack2, DACK, TNK1, ARK1, DPR2 or Kos1. In certain embodiments, the non-receptor tyrosine kinase may be BTK (Bruton's tyrosine kinase), ITK/EMT/TSK (interleukin 2-inducible T-cell kinase), RLK/TXK (tyrosine-protein kinase), BMX/ETK (bone marrow tyrosine kinase on chromosome) or Tec (tyrosine kinase expressed in hepatocellular carcinoma). In certain embodiments, the non-receptor tyrosine kinase may be Blk, Fgr, Fyn, Hck, Lck, Lyn, c-Src, c-Yes, Yrk, Frk (also known as Rak) or Srm. In certain embodiments, the small molecule inhibitor of a non-receptor tyrosine kinase may be a naturally occurring molecule. In certain embodiments, the small molecule inhibitor of a non-receptor tyrosine kinase may be Nintedanib, Imatinib, Dasatinib, gefitinib, erlotinib, or Lapatinib. In certain embodiments, the anti-inflammatory agent is a small molecule inhibitor of Nuclear factor kappa B (NFkB) p50, AKT, extracellular-signal-regulated kinase (ERK), I kappa B kinase gamma (IKKg), Toll Like Receptor 4 (TLR4), Toll Like Receptor 2 (TLR2), Signal transducer and activator of transcription 3 (STAT3), or interleukin-1 receptor-associated kinase 4 (IRAK). In certain embodiments, the small molecule is an activator of the glucocorticoid receptor. A small molecule activator of the glucocorticoid receptor (GR) may be:
1. Chloroprednisone=6α-chloro-17α,21-dihydroxypregna-1,4-diene-3,11,20-trione
2. Cloprednol=6-chloro-11β,17α,21-trihydroxypregna-1,4,6-triene-3,20-dione
3. Difluprednate=6α,9α-difluoro-11β,17α,21-trihydroxypregna-1,4-diene-3,20-dione 17α-butyrate 21-acetate
4. Fludrocortisone=9α-fluoro-11β,17α,21-trihydroxypregn-4-ene-3,20-dione
5. Fluocinolone=6α,9α-difluoro-11β,16α,17α,21-tetrahydroxypregna-1,4-diene-3,20-dione
6. Fluperolone=9α-fluoro-11β,17α,21-trihydroxy-21-methylpregna-1,4-diene-3,20-dione
7. Fluprednisolone=6α-fluoro-11β,17α,21-trihydroxypregna-1,4-diene-3,20-dione
8. Loteprednol=11β,17α,dihydroxy-21-oxa-21-chloromethylpregna-1,4-diene-3,20-dione
9. Methylprednisolone=6α-methyl-11β,17α,21-trihydroxypregna-1,4-diene-3,20-dione
10. Prednicarbate=11β,17α,21-trihydroxypregna-1,4-diene-3,20-dione 17α-ethylcarbonate 21-propionate
11. Prednisolone=11β,17α,21-trihydroxypregna-1,4-diene-3,20-dione
12. Prednisone=17α,21-dihydroxypregna-1,4-diene-3,11,20-trione
13. Tixocortol=11 β,17α-dihydroxy-21-sulfanylpregn-4-ene-3,20-dione
14. Triamcinolone=9α-fluoro-11β,16α,17α,21-tetrahydroxypregna-1,4-diene-3,20-dione
15. Alclometasone=7α-chloro-11β,17α,21-trihydroxy-16α-methylpregna-1,4-diene-3,20-dione
16. Beclometasone=9α-chloro-11β,17α,21-trihydroxy-16β-methylpregna-1,4-diene-3,20-dione
17. Betamethasone=9α-fluoro-11β,17α,21-trihydroxy-16α-methylpregna-1,4-diene-3,20-dione
18. Clobetasol=9α-fluoro-11β,17α-dihydroxy-16β-methyl-21-chloropregna-1,4-diene-3,20-dione
19. Clobetasone=9α-fluoro-16β-methyl-17α-hydroxy-21-chloropregna-1,4-diene-3,11,20-trione
20. Clocortolone=6α-fluoro-9α-chloro-11β,21-dihydroxy-16α-methylpregna-1,4-diene-3,20-dione
21. Desoximetasone=9α-fluoro-11β,21-dihydroxy-16α-methylpregna-1,4-diene-3,20-dione
22. Dexamethasone=9α-fluoro-11β,17α,21-trihydroxy-16α-methylpregna-1,4-diene-3,20-dione
23. Diflorasone=6α,9α-difluoro-11β,17α,21-trihydroxy-16β-methylpregna-1,4-diene-3,20-dione
24. Difluocortolone=6α,9α-difluoro-11β,21-dihydroxy-16α-methylpregna-1,4-diene-3,20-dione
25. Fluclorolone=6α-fluoro-9α,11β-dichloro-16α,17α,21-trihydroxypregna-1,4-dien-3,20-dione
26. Flumetasone=6α,9α-difluoro-11β,17α,21-trihydroxy-16α-methylpregna-1,4-diene-3,20-dione
27. Fluocortin=6α-fluoro-11β,21-dihydroxy-16α-methylpregna-1,4-diene-3,20,21-trione
28. Fluocortolone=6α-fluoro-11β,21-dihydroxy-16α-methylpregna-1,4-diene-3,20-dione
29. Fluprednidene=9α-fluoro-11β,17α,21-trihydroxy-16-methylenepregna-1,4-diene-3,20-dione
30. Fluticasone=6α,9α-difluoro-11β,17α-dihydroxy-16α-methyl-21-thia-21-fluoromethylpregna-1,4-dien-3,20-dione
31. Fluticasone furoate=6α,9α-difluoro-11β,17α-dihydroxy-16α-methyl-21-thia-21-fluoromethylpregna-1,4-dien-3,20-dione 17α-(2-furoate)
32. Halometasone=2-chloro-6α,9α-difluoro-11β,17α,21-trihydroxy-16α-methylpregna-1,4-diene-3,20-dione
33. Meprednisone=16β-methyl-17α,21-dihydroxypregna-1,4-diene-3,11,20-trione
34. Mometasone=9α,21-dichloro-11β,17α-dihydroxy-16α-methylpregna-1,4-diene-3,20-dione
35. Mometasone furoate=9α,21-dichloro-11β,17α-dihydroxy-16α-methylpregna-1,4-diene-3,20-dione 17α-(2-furoate)
36. Paramethasone=6α-fluoro-11β,17α,21-trihydroxy-16α-methylpregna-1,4-diene-3,20-dione
37. Prednylidene=11β,17α,21-trihydroxy-16-methylenepregna-1,4-diene-3,20-dione
38. Rimexolone=11β-hydroxy-16α,17α,21-trimethylpregna-1,4-dien-3,20-dione
39. Ulobetasol (halobetasol)=6α,9α-difluoro-11β,17α-dihydroxy-16β-methyl-21-chloropregna-1,4-diene-3,20-dione
40. Amcinonide=9α-fluoro-11β,16α,17α,21-tetrahydroxypregna-1,4-diene-3,20-dione cyclic 16α,17α-acetal with cyclopentanone, 21-acetate
41. Budesonide=11β,16α,17α,21-tetrahydroxypregna-1,4-diene-3,20-dione cyclic 16α,17α-acetal with butyraldehyde
42. Ciclesonide=11β,16α,17α,21-tetrahydroxypregna-1,4-diene-3,20-dione cyclic 16α,17α-acetal with (R)-cyclohexanecarboxaldehyde, 21-isobutyrate
43. Deflazacort=11β,21-dihydroxy-2′-methyl-5′H-pregna-1,4-dieno[17,16-d]oxazole-3,20-dione 21-acetate
44. Desonide=11β,16α,17α,21-tetrahydroxypregna-1,4-diene-3,20-dione cyclic 16α,17α-acetal with acetone
45. Formocortal (fluoroformylone)=3-(2-chloroethoxy)-9α-fluoro-11β,16α,17α,21-tetrahydroxy-20-oxopregna-3,5-diene-6-carboxaldehyde cyclic 16α,17α-acetal with acetone, 21-acetate
46. Fluclorolone acetonide (flucloronide)=6α-fluoro-9α,11β-dichloro-16α,17α,21-trihydroxypregna-1,4-dien-3,20-dione cyclic 16α,17α-acetal with acetone
47. Fludroxycortide (flurandrenolone, flurandrenolide)=6α-fluoro-11β,16α,17α,21-tetrahydroxypregn-4-ene-3,20-dione cyclic 16α,17α-acetal with acetone
48. Flunisolide=6α-fluoro-11β,16α,17α,21-tetrahydroxypregna-1,4-diene-3,20-dione cyclic 16α,17α-acetal with acetone
49. Fluocinolone acetonide=6α,9α-difluoro-11β,16α,17α,21-tetrahydroxypregna-1,4-diene-3,20-dione cyclic 16α,17α-acetal with acetone
50. Fluocinonide=6α,9α-difluoro-11β,16α,17α,21-tetrahydroxypregna-1,4-diene-3,20-dione cyclic 16α,17α-acetal with acetone, 21-acetate
51. Halcinonide=9α-fluoro-11β,16α,17α-trihydroxy-21-chloropregn-4-ene-3,20-dione cyclic 16α,17α-acetal with acetone
52. Triamcinolone acetonide=9α-fluoro-11β,16α,17α,21-tetrahydroxypregna-1,4-diene-3,20-dione cyclic 16α,17α-acetal with acetone
53. Cortivazol=6,16α-dimethyl-11β,17α,21-trihydroxy-2′-phenyl[3,2-c]pyrazolopregna-4,6-dien-20-one 21-acetate
54. RU-28362=6-methyl-11β,17β-dihydroxy-17α-(1-propynyl)androsta-1,4,6-trien-3-one

In certain embodiments, the anti-inflammatory agent is a large molecule. In certain embodiments, the large molecule is a peptide. The peptide may be an inhibitor of Myd88. A peptide inhibitor of Myd88 may include a portion of the Myd88 protein involved in heterodimerization of Myd88 and may reduce the heterodimerization. In certain embodiments, the peptide inhibitor of Myd88 may include the sequence DRQIKIWFQNRRMKWKKRDVLPGT (SEQ ID NO:1) or a fragment thereof. In certain embodiments, the peptide inhibitor of Myd88 may include the sequence RDVLPGT (SEQ ID NO:2).

In certain embodiments, the anti-inflammatory agent is a large molecule inhibitor of Nuclear factor kappa B (NFkB) p50, AKT, extracellular-signal-regulated kinase (ERK), I kappa B kinase gamma (IKKγ), Toll Like Receptor 4 (TLR4), Toll Like Receptor 2 (TLR2), Signal transducer and activator of transcription 3 (STAT3), or interleukin-1 receptor-associated kinase 4 (IRAK). In certain embodiments, the large molecule is a peptide. In certain embodiments, the peptide is an inhibitor of NFkB p50. In certain embodiments, the peptide inhibitor of NFkB p50 binds to NFkB p50. In certain embodiments, the peptide inhibitor of NFkB p50 comprises the amino acid sequence VQRKRQKLM (SEQ ID NO:3). In certain embodiments, the peptide is an inhibitor of AKT. In certain embodiments, the peptide inhibitor of AKT comprises the amino acid sequence AVTDHPDRLWAWEKF (SEQ ID NO:4). In certain embodiments, the peptide is an inhibitor of ERK. In certain embodiments, the peptide inhibitor of ERK comprises the amino acid sequence MPKKKPTPIQLNP (SEQ ID NO:5). In certain embodiments, the peptide is an inhibitor of IKKγ. In certain embodiments, the peptide inhibitor of IKKγ comprises the amino acid sequence TALDWSWLQTE (SEQ ID NO:6). In certain embodiments, the peptide is an inhibitor of TLR4. In certain embodiments, the peptide inhibitor of TLR4 comprises the amino acid sequence KYSFKLILAEY (SEQ ID NO:7) or PGFLRDPWCKYQML (SEQ ID NO:8). In certain embodiments, the peptide is an inhibitor of TLR2. In certain embodiments, the peptide inhibitor of TLR2 comprises the amino acid sequence PGFLRDPWCKYQML (SEQ ID NO:8). In certain embodiments, the peptide is an inhibitor of STAT3. In certain embodiments, the peptide inhibitor of STAT3 comprises the amino acid sequence PYLKTKAAVLLPVLLAAP (SEQ ID NO:9). In certain embodiments, the peptide is an inhibitor of IRAK. In certain embodiments, the peptide inhibitor of IRAK comprises the amino acid sequence KKARFSRFAGSSPSQSSMVAR (SEQ ID NO:10). It is understood that reference to a peptide having a particular sequence also includes peptides having a sequence that is a variant of the disclosed sequence, such as, shorter or longer sequences (+/−1, 2, 3, 4, or 5 amino acids) and/or sequences having conservative substitutions.

In certain embodiments, exgNVs may be enriched in membrane proteins, such as, proteins localized in the plasma membrane, e.g., transport proteins. “Enriched” in the context of a component enriched in the exgNVs disclosed herein means that the enriched component is present in the exgNVs at a higher concentration by total weight, e.g., at least a three-fold greater concentration by total weight, e.g., at least 5-fold greater concentration, at least 10-fold greater concentration, at least 30-fold greater concentration, at least 50-fold greater concentration, or at least 100-fold greater concentration than the concentration of that component by total weight in NVs from which the exgNVs are derived.

The exgNVs are prepared by a process that includes serial extrusion of a human cell followed by exposure to high pH prior to forming the exgNVs. The exgNVs comprise a reduced amount of one or more of cytoplasmic proteins, lysosomal proteins, exosomal proteins, plasma membrane proteins, and endoplasmic reticulum proteins as compared to vesicles made from the same type of cell by exposure to high pH in absence of an extrusion step. Such vesicles prepared without extruding cells are referred to herein as gNVs. Extrusion step refers to the step of passing cells through a membrane or a filter with pore size smaller than the size of the cells which step results in breaking of the cells into smaller pieces which pieces include vesicles. Serial extrusion is a type of extrusion where cells are passed through a series of membranes with increasingly smaller pore size such that the pieces of the cells get progressively smaller as they are forced through the smaller pores. In certain embodiments, serial extrusion involves passing cells through a filter comprising pores having an average diameter of 10 um; passing the matter filtered through the 10 um filter through a filter comprising pores having an average diameter of 5 um; and passing the matter filtered through 5 um filter through filter comprising pores having an average diameter of 1 um.

The amount of cytoplasmic proteins present in exgNVs may be 70% (or lower, e.g., 60% or 50%) of the amount of cytoplasmic proteins present in gNVs produced without extrusion of the cells. The amount of lysosomal proteins present in exgNVs may be 70% (or lower, e.g., 60% or 50%) of the amount of lysosomal proteins present in gNVs produced without extrusion of the cells. The amount of exosomal proteins present in exgNVs may be 70% (or lower, e.g., 60% or 50%) of the amount of exosomal proteins present in gNVs produced without extrusion of the cells. The amount of plasma membrane proteins present in exgNVs may be 70% (or lower, e.g., 60% or 50%) of the amount of plasma membrane proteins present in gNVs produced without extrusion of the cells. The amount of endoplasmic reticulum proteins present in exgNVs may be 70% (or lower, e.g., 60% or 50%) of the amount of endoplasmic reticulum proteins present in gNVs produced without extrusion of the cells.

gNVs can be prepared by directly exposing the cells to alkaline pH, followed by density gradient ultracentrifugation; isolation of the open sheet of membranes; and sonication of the membrane sheets to generate the gNVs. Thus, different from the generation of exgNVs, generation of gNVs does not involve extrusion of the cells prior to exposure to high pH.

In certain embodiments, the exgNVs are prepared by a process that comprises serial extrusion of a cell followed by exposure to high pH prior to forming the exgNVs and where the exgNVs comprise an enrichment of mitochondrial proteins and a reduced amount of cytoplasmic proteins as compared to vesicles made from the same type of cell by exposure to high pH in absence of an extrusion step. The reduced amount may be 70% or less (e.g., 60% or 50%) of the cytoplasmic proteins as compared to gNVs prepared without extrusion of cells. The enrichment of mitochondrial proteins may be an increase of at least 0.5×, 1×, 2× or more of the amount of mitochondrial proteins present in gNVs prepared without extrusion of cells.

In certain embodiments, exgNVs are prepared by a process that comprises serial extrusion of a cell followed by exposure to high pH prior to forming the exgNVs and wherein the exgNVs reduce TNF-α production while gNVs made from the same type of cell by exposure to high pH in absence of an extrusion step do not significantly reduce TNF-α production. In certain embodiments, the gNVs prepared without extrusion of cells may not result is a significant reduction in TNF-α level produced due to administration of OMVs while the exgNVs result in a significant reduction in TNF-α level produced due to administration of the OMVs. The OMVs may be OMVs generated from a gram-negative bacterium such as E. coli or P. aeruginosa.

In certain embodiments, compositions that include the exgNVs or the SyEVs loaded with an anti-inflammatory agent as described herein are provided. The compositions may include the exgNVs or the SyEVs loaded with an anti-inflammatory agent and a carrier, diluent, vehicle, excipient, and the like. In certain embodiments, the compositions of the present disclosure may include the exgNVs loaded with an anti-inflammatory agent and a pharmaceutically acceptable carrier, diluent, vehicle, excipient, and the like. In certain embodiments, the compositions may further include an additional prophylactic or therapeutic agent. In certain embodiments, the compositions may include exgNVs or the SyEVs in an amount effective for reducing inflammation in a subject in need thereof. In certain embodiments, the amount of anti-inflammatory agent loaded exgNVs that provide a therapeutic effect is less than the amount of exgNVs needed to provide the same effect when the exgNVs are not loaded with the anti-inflammatory agent. In certain embodiments, the amount of anti-inflammatory agent loaded exgNVs that provide a therapeutic effect is less than the amount of the anti-inflammatory agent needed to provide the same effect when the anti-inflammatory agent is not loaded in the exgNVs. Compositions may include exgNVs or the SyEVs derived from different cells and/or loaded with different anti-inflammatory agents. For example, the exgNVs or the SyEVs may be derived from two, three, four, or more different types of cells. In certain embodiments, the composition may include a first type of exgNVs that includes a first anti-inflammatory agent and a second type of exgNVs that includes a second anti-inflammatory agent, and so on.

In certain embodiments, the composition includes exgNVs or the SyEVs generated from human mesenchymal stem cells and loaded with an anti-inflammatory agent that is an inhibitor of Myd88, a non-RTK, NFkB p50, AKT, ERK, IKKg, TLR4, TLR2, STAT3, or IRAK, or an activator of glucocorticoid receptor or CsA.

A carrier, diluent, vehicle, excipient, and the like may be salt, buffer, antioxidant (e.g., ascorbic acid and sodium bisulfate), preservative (e.g., benzyl alcohol, methyl parabens, ethyl or n-propyl, p-hydroxybenzoate), emulsifying agent, suspending agent, dispersing agent, solvent, filler, bulking agent, detergent, and/or adjuvant. For example, a suitable vehicle may be physiological saline solution or buffered saline, possibly supplemented with other materials common in pharmaceutical compositions for, e.g., parenteral administration. Neutral buffered saline or saline mixed with serum albumin are further exemplary vehicles. Those skilled in the art will readily recognize a variety of buffers that could be used in the compositions. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS). In certain embodiments, an adjuvant included in the disclosed compositions may be poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, acrylic or methacrylic polymers, or copolymers of maleic anhydride and Aquila's QS21 stimulon.

The exgNVs may be enclosed in a device that may be used for administering the exgNVs. The SyEVs may be enclosed in a device that may be used for administering the SyEVs. In certain embodiments, the device may be a thin film device comprising a biocompatible matrix. The biocompatible matrix may be a scaffold that may release the exgNVs or SyEVs upon placement of the device in a subject, e.g., by wetting of the matrix. The matrix may be made from a biocompatible polymer, e.g., polydimethylsiloxane monoacrylate or polydimethylsiloxane monomethacrylate. In certain embodiments, the matrix is a silicone elastomer.

In certain embodiments, the exgNVs or SyEVs loaded with an anti-inflammatory agent and compositions thereof find use in a method for reducing at least one proinflammatory cytokine in a subject in need thereof, the method comprising administering the composition to the subject. Such methods are described in detail in the following section.

### Methods

In certain embodiments, a method for treating a subject in need thereof is provided. In certain embodiments, a method for reducing inflammation in a subject in need thereof is provided. The method may include administering to the subject an effective amount of the ghost nanovesicles (e.g., exgNVs or SyEVs) comprising an anti-inflammatory agent, where the exgNVs have an anti-inflammatory property, enhance anti-inflammatory effect of the anti-inflammatory agent, and are produced from a human cell, and wherein the anti-inflammatory agent is present in a therapeutically effective amount, wherein the gNVs reduce the levels of at least one proinflammatory cytokine in the subject.

The term “reduced” in the context of inflammatory response means production of a lower level of a proinflammatory cytokine in the presence of the exgNVs as compared to that produced in absence of the exgNVs. In some embodiments, production of cytokines is lowered by at least 5%, for example, at least 10%, at least 20%, at least 30%, at least 40%, at least 50%, at least 60% or more as compared to that produced in absence of administration of exgNVs. The at least one proinflammatory cytokine may include one or more of IL-2, IL-4, IL-6, IL-12, IL-12p70, IL-17, tumor necrosis factor alpha (TNF-α), or interferon gamma (IFN-γ). The human cell from which the exgNVs or SyEVs are derived may be as described in the preceding section providing description of the exgNVs or SyEVs of the present disclosure. In certain embodiments, the human cell is a mesenchymal stem cell.

In certain embodiments, the exgNVs may be made by a method that includes disrupting (e.g., by serial extrusion) the human cell to generate vesicles; separating the vesicles based on density and isolating nanovesicles; exposing the isolated nanovesicles to an alkaline pH to open the nanovesicles thereby generating plasma membrane sheets; purifying the plasma membrane sheets; adding a therapeutic agent to the purified membrane sheets; and applying energy to the purified plasma membrane sheets sufficient to convert the plasma membrane sheets into exgNVs comprising the anti-inflammatory agent. As used herein, the term “gNVs loaded with an anti-inflammatory agent” or “anti-inflammatory agent loaded gNVs” refer to exgNVs that comprise the anti-inflammatory agent. Depending on the type of anti-inflammatory agent, the agent may be present in the lumen and/or on the surface of the exgNVs.

In certain embodiments, disrupting the human cell to generate vesicles may involve mechanical, electrical or chemical methods for cytolysis. Examples of techniques for cytolysis include osmosis, electroporation, sonication, homogenization, detergent treatment, freeze-thawing, extrusion, mechanical degradation, and chemical treatment, but are not limited thereto. In certain embodiments, the human cell is not disrupted by detergent treatment. In a mechanical degradation method, a solution of human cells is shaken together with metal, ceramic or sufficiently hard plastic balls. In certain embodiments, disrupting the mammalian cell may include applying a shear force to the human cell. Shear force may be applied by extruding the human cell. Extrusion may include forcing the human cells through pores smaller than the size of the mammalian cells. In the context of extrusion, mammalian cells may be forced to sequentially pass through a series of filters having decreasing pore sizes. For example, mammalian cells are sequentially passed through three filters with respective pore sizes of 10 μm→5 μm→1 μm to form vesicles.

In certain embodiments, disrupting the human cell may include applying acoustic energy to the human cell. Acoustic energy may be applied via a sonication device. Sonication conditions may be adjusted for the desired disruptive energy. For example, low temperature, low energy, and/or short duration for sonication may be used when disrupting spheroplasts to generate vesicles. Sonication can be performed with different degree of intensity, including low energy sonication over periods of 1 minute to 3 hours. In certain embodiments, sonication may be performed using an ultrasonic probe-type device. In certain embodiments, an ultrasonic bath may be used for sonication. The duration of sonication may be adjusted based on the type of device being used to perform the sonication. For example, an ultrasonic probe-type device may provide about 1000 times higher energy than an ultrasonic bath. In certain embodiments, ultrasonic probe-type device may be used for disrupting the mammalian cell.

Following disruption of the human cells to generate vesicles, such as, vesicles that have the plasma membrane enclosing cytosolic contents, these vesicles may be isolated from any remaining human cells. Separation of these vesicles from human cells may be performed using differences in size, density, buoyancy, etc. In certain embodiments, centrifugation (e.g., density gradient centrifugation or density gradient ultracentrifugation) or filtration may be performed to isolate the vesicles. In certain embodiments, the vesicles may be purified using density gradient ultracentrifugation, where vesicles present in between 10% and 50% density gradient may be isolated. The vesicles present in between 10% and 50% density gradient are mostly nanometer sized vesicles or nanovesicles.

The isolated nanovesicles may then be exposed to an alkaline solution to open up the nanovesicles which expels the cytoplasmic content of the nanovesicles. In certain embodiments, the alkaline solution used for opening the nanovesicles may have a pH of 11-14. An alkaline solution for opening the nanovesicles may be prepared a sodium carbonate (Na₂CO₃), sodium hydroxide (NaOH), ammonia (NH₃), calcium hydroxide (Ca(OH)₂), potassium hydroxide (KOH), sodium hydrogen carbonate (NaHCO₃), or magnesium hydroxide (Mg(OH)₂) solution. The duration of incubation of the nanovesicles in an alkaline solution may be adjusted based on the number of nanovesicles, total volume of the solution, and the like. As used herein the step of incubating or exposing vesicles to an alkaline pH may include using an alkaline solution having a pH of 9-14, e.g., pH of 10-14, pH of 11-14, pH of 12-14, or pH of 13-14.

Plasma membrane sheets generated from opening of nanovesicles may be separated from whole nanovesicles (i.e., unopened) by utilizing any suitable separation method. In certain embodiments, purifying the plasma membrane sheets may involve centrifugation, e.g., centrifugation (such as, density gradient centrifugation or density gradient ultracentrifugation), filtration, or another suitable method, such as size exclusion, dialysis, tangential flow filtration and the like. In certain embodiments, the plasma membrane sheets may be purified using density gradient ultracentrifugation, where plasma membrane sheets present in between 10% and 30% density gradient may be isolated. The plasma membrane sheets present in between 10% and 30% density gradient are substantially free of nanovesicles.

In certain embodiments, the method of generating the exgNVs may be involve applying energy or force to the purified plasma membrane sheets sufficient to convert the plasma membrane sheets into exgNVs. Suitable sources of energy include mild sonication, shear force, acoustic force, freeze-thaw, and the like. In certain embodiments, the purified plasma membrane sheets may be sonicated for a duration of time sufficient to convert the plasma membrane sheets into exgNVs. In certain embodiments, the purified plasma membrane sheets may be sonicated by applying energy 100-1000 times less than that applied for disrupting mammalian cells. In certain embodiments, mild sonication may include using an ultrasonic bath for converting the plasma membrane sheets into exgNVs.

In certain embodiments, the method of generating the SyEVs may include isolating exosomes released by eukaryotic cells. Separation of exosomes from eukaryotic cells may be performed using differences in size, density, buoyancy, etc. In certain embodiments, centrifugation (e.g., density gradient centrifugation or density gradient ultracentrifugation) or filtration may be performed to isolate the exosomes. In certain embodiments, the exosomes may be purified using density gradient ultracentrifugation, where vesicles present in between 10% and 50% density gradient may be isolated. The exosomes present in between 10% and 50% density gradient are mostly nanometer sized.

The isolated exosomes may then be exposed to a mildly alkaline solution to open up the exosomes which expels the cytoplasmic content of the exosomes. In certain embodiments, the alkaline solution used for opening the exosomes may have a pH more than 7 to less than 10. An alkaline solution for opening the exosomes may be prepared a sodium carbonate (Na₂CO₃), sodium hydroxide (NaOH), ammonia (NH₃), calcium hydroxide (Ca(OH)₂), potassium hydroxide (KOH), sodium hydrogen carbonate (NaHCO₃), or magnesium hydroxide (Mg(OH)₂) solution. The duration of incubation of the exosomes in the alkaline solution may be adjusted based on the number of nanovesicles, total volume of the solution, and the like. As used herein the step of incubating or exposing exosomes to an alkaline pH may include using an alkaline solution having a pH of 7.2-9.8, e.g., pH of 7.5-9.5, pH of 8-9.5, pH of 8.5-9.5, or pH of 9.

Plasma membrane sheets generated from opening of exosomes may be separated from whole exosomes (i.e., unopened) by utilizing any suitable separation method. In certain embodiments, purifying the plasma membrane sheets may involve centrifugation, e.g., centrifugation (such as, density gradient centrifugation or density gradient ultracentrifugation), filtration, or another suitable method, such as size exclusion, dialysis, tangential flow filtration and the like. In certain embodiments, the plasma membrane sheets may be purified using density gradient ultracentrifugation, where plasma membrane sheets present in between 10% and 30% density gradient may be isolated. The plasma membrane sheets present in between 10% and 30% density gradient are substantially free of exosomes.

In certain embodiments, the method of generating the syEVs may be involve applying energy or force to the purified plasma membrane sheets sufficient to convert the plasma membrane sheets into SyEVs. Suitable sources of energy include mild sonication, shear force, acoustic force, freeze-thaw, and the like. In certain embodiments, the purified plasma membrane sheets may be sonicated for a duration of time sufficient to convert the plasma membrane sheets into SyEVs. In certain embodiments, the purified plasma membrane sheets may be sonicated by applying energy 100-1000 times less than that applied for disrupting mammalian cells. In certain embodiments, mild sonication may include using an ultrasonic bath for converting the plasma membrane sheets into SyEVs.

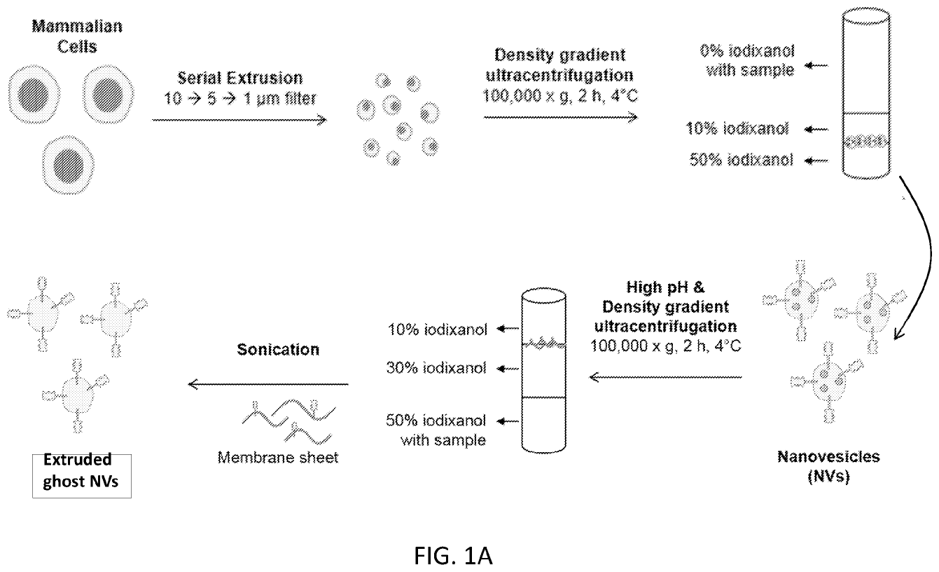

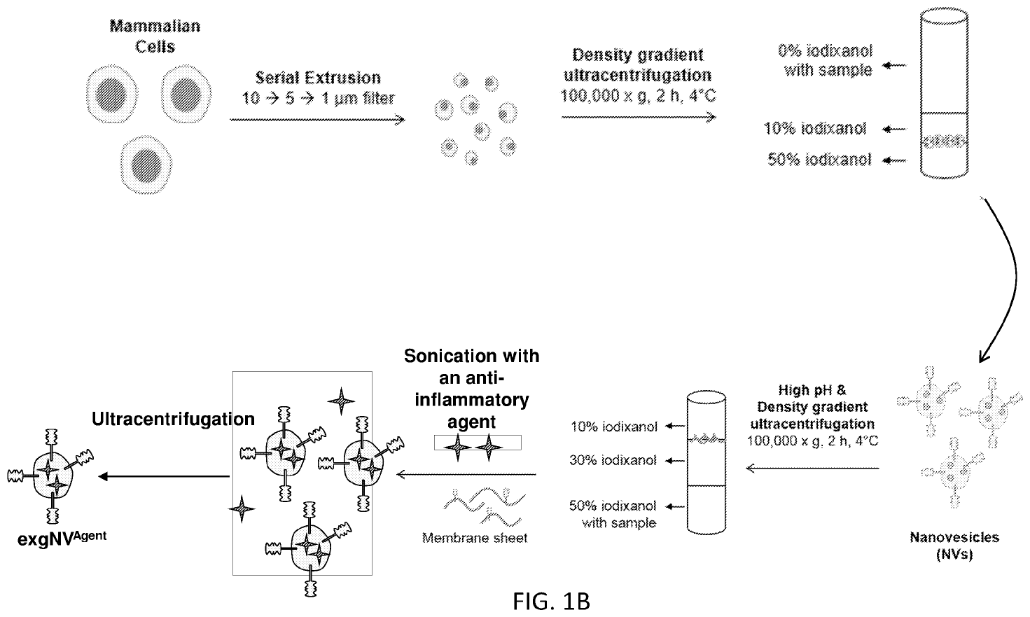

In certain embodiments, the exgNVs of the present disclosure may be prepared by the method depicted in FIG. 1A. The loading of the exgNVs may be performed as depicted in FIG. 1B. As shown in FIG. 1A, a mammalian cell may be disrupted by serial extrusion through filters of increasingly small pores, forcing the cells to break into vesicles. Separating the vesicles based on size using density gradient ultracentrifugation using a density gradient of from 0% to 50% iodixanol. Isolating nanovesicles present between 10% and 50% density layers; exposing the nanovesicles to an alkaline solution (e.g., pH11-pH14) to open the NVs; separating the opened NVs using density gradient ultracentrifugation by using a density gradient of from 10% to 50% iodixanol. Isolating opened NVs (i.e., membrane sheets) present between 10%-30% density layers; and sonicating the isolated membrane sheets to generate exgNVs. As shown in FIG. 1B, the isolated membrane sheets are mixed with an anti-inflammatory agent and the mixture is sonicated to generate exgNVs loaded with the anti-inflammatory agent.

As shown in FIG. 14A, SyEVs prepared from human cells genetically modified to express LFA-1 show higher binding affinity for target cells when the SyEVs are prepared using a mild alkaline pH, such as pH9, as compared to SyEVs prepared using a higher alkaline pH of pH10 or pH11. In certain embodiments, the alkaline pH may be less than pH10, e.g., pH7.5-pH9.5, such as, pH8-pH9.5, pH8.5-pH9.5, pH8.5-pH9, for example, pH9.

The subject in need of reduction of inflammation who is treated by the exgNVs or the SyNVs of the present disclosure may have or may be susceptible to developing an inflammatory related condition. The inflammatory related condition may be cancer, multiple sclerosis, psoriasis, dry eye disease, asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory condition, demyelinating polyneuropathy, pancreatitis, psoriatic arthritis, periodontitis, Behcet's disease, sinusitis, polymyalgia rheumatic, systemic lupus erythematous, fibromyalgia, dermatitis, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, neurodegenerative disease, Parkinson's disease, Alzheimer's disease, amyotrophic lateral sclerosis (ALS), cerebellar ataxias, spinobulbar muscular atrophy (SBMA), spinomuscular atrophy (SMA), lysosomal storage diseases, cerebral palsy, glioma, glioblastoma, muscular dystrophy, ataxia telangiectasia (AT), schizophrenia, depression, bipolar disorder, attention deficit disorder, or trisomy 21. In certain embodiments, the inflammatory related condition may be asthma. In certain embodiments, the inflammatory related condition may be sepsis. In certain embodiments, the inflammatory related condition may be infection. In certain embodiments, the inflammatory related condition may be bacterial, viral or parasitic infection.

### Administration of ExgNVs or SyEVs

The present disclosure contemplates the administration of the disclosed compositions in any appropriate manner for prevention and/or treatment of a condition as described herein. Suitable routes of administration include parenteral (e.g., intramuscular, intravenous, intraarterial, subcutaneous (e.g., injection), intraperitoneal, intracisternal, intraarticular, intraperitoneal, intracerebral (intraparenchymal and intracerebroventricular), intrathecal, oral, nasal, vaginal, sublingual, intraocular, rectal, topical (e.g., transdermal), sublingual and inhalation, as well as injection directly into a diseased tissue, for example a tumor tissue.

In certain embodiments, the administering comprises local administration to a target site in the subject. In certain embodiments, the target site comprises or is susceptible to developing an inflammatory response. In certain embodiments, the target site has an injury. The target site may be adjacent to a site that has injury. The target site may include a site in the central nervous system. The target site may be brain. The target site may have an arterial blockage. In certain embodiments, the administering may be intraarterial administering at the site of arterial blockage, e.g., a catheter used for clot retrieval may be used to administer the exgNVs or SyEVs comprising an anti-inflammatory agent after clot retrieval.

In certain embodiments, the compositions of exgNVs or SyEVs comprising an anti-inflammatory agent may be injected into or adjacent a tumor. In certain embodiments, a composition of an anticancer agent and a composition of the exgNVs or SyEVs comprising an anti-inflammatory agent may be administered simultaneously to a subject.

The present disclosure contemplates methods wherein the compositions of the present disclosure is administered to a subject at least twice daily, at least once daily, at least once every 48 hours, at least once every 72 hours, at least once weekly, at least once every 2 weeks, or once monthly.

The compositions of the present disclosure may be administered acutely or continuously. In certain embodiments, a composition provided herein may be administered by continuous infusion. An Omaya chamber may be used to continuously infuse the exgNVs into the cerebral ventricle of a subject in need thereof. Similarly, an osmotically driven pump or another continuous infusion system might be used to deliver the compositions of the present disclosure to various tissues, fluids, organs, or compartments.

### Combination Therapy

The present disclosure contemplates the use of the compositions provided herein in combination with one or more active therapeutic agents or other prophylactic or therapeutic modalities. In such combination therapy, the various active agents frequently have different mechanisms of action. Such combination therapy may be especially advantageous by allowing a dose reduction of one or more of the agents, thereby reducing or eliminating the adverse effects associated with one or more of the agents; furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, compositions of the present disclosure are administered or applied sequentially, e.g., where one agent is administered prior to one or more other agents. In other embodiments, the compositions are administered simultaneously, e.g., where two or more compositions are administered at or about the same time; the two or more compositions may be present in two or more separate formulations or combined into a single formulation (i.e., a co-formulation). Regardless of whether the two or more compositions are administered sequentially or simultaneously, they are considered to be administered in combination for purposes of the present disclosure.

The compositions of the present disclosure can be used in combination with other agents useful in the treatment, prevention, suppression or amelioration of the diseases, disorders or conditions set forth herein, including those that are normally administered to subjects suffering from inflammation.

Also provided herein are compositions comprising the exgNVs or SyEVs complexed with a Cas polypeptide and a guide RNA. In certain embodiments, the exgNVs or SyEVs are complexed with a Cas polypeptide and a guide RNA via an amphipathic molecule. The amphipathic molecule may include a lipophilic portion and a hydrophilic polymer, such as, a Cholesterol functionalized poly(ethylene glycol), e.g., Chol-PEG50, Chol-PEG200, Chol-PEG600, etc.

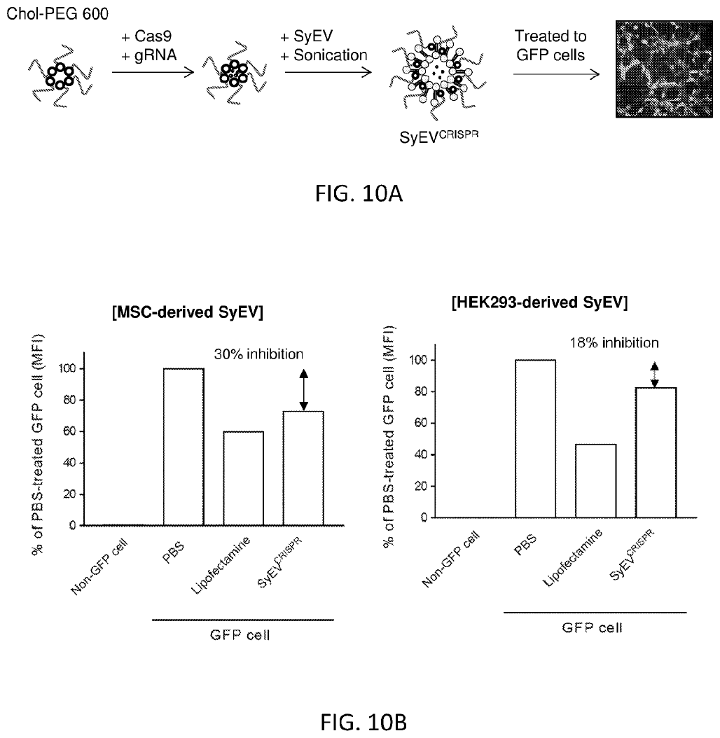

In certain embodiments, the SyEVs complexed with a Cas polypeptide and a guide RNA may be prepared by a method depicted in FIG. 10A. In certain embodiments, the SyEVs may be prepared from MSCs. FIG. 10B shows that SyEVs prepared from MSCs are surprisingly more efficient at delivering Cas polypeptide and guide RNA to cells as compared to SyEVs prepared from HEK293 cells.

### ExgNVs or SyEVs Comprising Lymphocyte Function-Associated Antigen-1 (LFA-1)

ExgNVs or SyEVs produced from a human cell line genetically modified to express lymphocyte function-associated antigen-1 (LFA-1), where LFA-1 is located on surface of the exgNVs or SyEVs are provided.

ExgNVs or SyEVs produced from a human cell line genetically modified to express Macrophage-1 antigen (Mac-1), where Mac-1 is located on surface of the NVs are provided.

The genetically modified human cell line may be an stem cell line (e.g., ES cell line or mesenchymal stem cell line), an immune cell line, or a non-immune cell line. Human embryonic kidney 293 cell line (HEK 293), 293F cell line, and 293T cell line are examples of a non-immune cell line. Jurkat cell line is derived from human T-cells and is an example of an immune cell line. Immune cell lines may be derived from T cells, monocytes, or dendritic cells. In certain embodiments, the genetically modified human cell line is not a cell line that naturally expressed LAF-1, e.g., Jurkat cells. In certain embodiments, the genetically modified human cell line is a non-immune cell line, such as, HEK 293 cell line, 293F cell line, or 293T cell line.

LFA-1 is a heterodimer composed of CD11a and CD18 and is expressed on surface of all leukocytes. LFA-1 plays a central role in leukocyte intercellular adhesion through interactions with its ligands, ICAMs 1-3 (intercellular adhesion molecules 1 through 3), and also functions in lymphocyte costimulatory signaling.

Mac-1 is a heterodimer is composed of CD11 b and CD18 and is expressed on surface of B and T lymphocytes, polymorphonuclear leukocytes (mostly neutrophils), NK cells, and mononuclear phagocytes like macrophages. Mac-1 can mediate binding to ICAM-1 (intercellular adhesion molecule 1).

The ExgNVs or SyEVs disclosed herein comprise on their surface LFA-1 or Mac-1 molecules in an amount sufficient to mediate binding of the NVs to cells expressing ICAM-1. Thus, the ExgNVs or SyEVs are targeted to ICAM-1 expressing cells and can deliver therapeutic agents to such cells.

The therapeutic agents can be small molecules or large molecules. The therapeutic agents can be anti-inflammatory agents, anti-cancer agents, antibiotics, gene editing agents (e.g., Cas polypeptide and guide RNA) and the like. The cells expressing ICAM-1 can be any cell in a subject in need for treatment. A non-limiting sample of diseases wherein the ExgNVs or SyEVs as per the present invention may be administered for treatment comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, non-alcoholic steatohepatitis (NASH), non-alcoholic fatty liver disease (NAFLD), fibrosis, Guillain-Barre syndrome, acute myocardial infarction, ARDS, sepsis, meningitis, encephalitis, liver failure, kidney failure, heart failure or any acute or chronic organ failure and the associated underlying etiology, graft-vs-host disease, Duchenne muscular dystrophy, Becker muscular dystrophy, and other muscular dystrophies, lysosomal storage diseases such as Gaucher disease, Fabry's disease, MPS 1, II (Hunter syndrome), and Ill, Niemann-Pick disease, cystinosis, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sezary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

### EXAMPLES OF NON-LIMITING EMBODIMENTS OF THE DISCLOSURE

Embodiments, including embodiments, of the present subject matter described above may be beneficial alone or in combination, with one or more other embodiments or embodiments. Without limiting the foregoing description, certain non-limiting embodiments of the disclosure are provided below. As will be apparent to those of skill in the art upon reading this disclosure, each of the individually numbered clauses may be used or combined with any of the preceding or following individually numbered clauses. This is intended to provide support for all such combinations of embodiments and is not limited to combinations of embodiments explicitly provided below:

1. Synthetic eukaryotic vesicles (SyEVs) generated from eukaryotic cells and comprising functional integrin on surface of the SyEVs and substantially devoid of cytoplasmic content of the eukaryotic cells.

2. The SyEVs of embodiment 1, wherein the SyEVs are generated by a method comprising:
isolating exosomes secreted by the eukaryotic cells;
opening the isolated exosomes by exposing them to a mild alkaline pH, wherein the mild alkaline pH is sufficient to cause opening of the exosomes while allowing retention of the functional integrin on the surface of the SyEVs; and
closing the exosomes by applying energy to the opened exosomes to generate the SyEVs.

3. The SyEVs of embodiment 2, wherein the mild alkaline pH comprises a pH of less than 10.

4. The SyEVs of embodiment 3, wherein the mild alkaline pH comprises pH8.5-9.5.

5. The SyEVs of any one of embodiments 2-4, wherein the method further comprises incubating the opened exosomes with a therapeutic agent.

6. The SyEVs of any one of embodiments 2-5, wherein applying energy comprises sonication.

7. The SyEVs of any one of embodiments 1-6, wherein the SyEVs comprise a therapeutic agent present inside the SyEVs.

8. The SyEVs of any one of embodiments 1-7, wherein the eukaryotic cells comprise human cells.

9. The SyEVs of embodiment 8, wherein the human cells comprise primary cells or a cell line.

10. The SyEVs of embodiment 9, wherein the primary cells comprise multipotent stem cells.

11. The SyEVs of embodiment 10, wherein the multipotent stem cells comprise mesenchymal stem cells.

12. The SyEVs of embodiment 9, wherein the cell line comprises embryonic stem cells, pluripotent stem cells, or an immortalized cell line.

13. The SyEVs of embodiment 12, wherein the immortalized cell line comprises HEK 293 cells.

14. The SyEVs of any one of embodiments 1-13, wherein the eukaryotic cells are genetically modified to express an integrin on the cell surface.

15. The SyEVs of embodiment 14, wherein the integrin is lymphocyte function-associated antigen-1 (LFA-1).

16. The SyEVs of embodiment 14, wherein the integrin is Macrophage-1 antigen (Mac-1).

17. The SyEVs of any one of embodiments 1-16, wherein the SyEVs comprise a therapeutic agent located on the surface of the SyEVs.

18. The SyEVs of embodiment 17, wherein the eukaryotic cells are genetically modified to express the therapeutic agent located on the surface of the SyEVs.

19. The SyEVs of any one of embodiments 5-18, wherein the therapeutic agent is a small molecule or a large molecule.

20. The SyEVs of embodiment 19, wherein the therapeutic agent is an anti-inflammatory agent.

21. The SyEVs of embodiment 20, wherein the anti-inflammatory agent is cyclosporine A, an inhibitor of Myeloid Differentiation Primary Response 88 (Myd88), an activator of the glucocorticoid receptor, an inhibitor of a non-receptor tyrosine kinase, an inhibitor of Nuclear factor kappa B (NFkB) p50, AKT, extracellular-signal-regulated kinase (ERK), I kappa B kinase gamma (IKKg), Toll Like Receptor 4 (TLR4), Toll Like Receptor 2 (TLR2), Signal transducer and activator of transcription 3 (STAT3), or interleukin-1 receptor-associated kinase 4 (IRAK).

22. The SyEVs of embodiment 21, wherein the inhibitor of MYD88 is a small molecule or a large molecule.

23. The SyEVs of embodiment 22, wherein the small molecule inhibitor of MYD88 is T6167923 or ST2825 and the large molecule inhibitor of MYD88 is a peptide, optionally, wherein the peptide comprises the amino acid sequence RDVLPGT (SEQ ID NO:2).

24. The SyEVs of embodiment 21, wherein the activator of the glucocorticoid receptor is a small molecule, the inhibitor of a non-receptor tyrosine kinase comprises Nintedanib, Imatinib, Dasatinib, gefitinib, erlotinib, or Lapatinib, the inhibitor of NFkB p50, AKT, ERK, IKKg, TLR4, TLR2, STAT3, or IRAK comprises a peptide.

25. The SyEVs of embodiment 24, wherein the inhibitor of NFkB p50 comprises a peptide comprising the amino acid sequence VQRKRQKLM (SEQ ID NO:3), the inhibitor of AKT comprises a peptide comprising the amino acid sequence AVTDHPDRLWAWEKF (SEQ ID NO:4), the inhibitor of ERK comprises a peptide comprising the amino acid sequence MPKKKPTPIQLNP (SEQ ID NO:5), the inhibitor of IKKg comprises a peptide comprising the amino acid sequence TALDWSWLQTE (SEQ ID NO:6), the inhibitor of TLR4 comprises a peptide comprising the amino acid sequence KYSFKLILAEY (SEQ ID NO:7), the inhibitor of TLR2 comprises a peptide comprising the amino acid sequence PGFLRDPWCKYQML (SEQ ID NO:8), the inhibitor of STAT3 comprises a peptide comprising the amino acid sequence PYLKTKAAVLLPVLLAAP (SEQ ID NO:9), or the inhibitor of IRAK comprises a peptide comprising the amino acid sequence KKARFSRFAGSSPSQSSMVAR (SEQ ID NO:10).

26. The SyEVs of embodiment 19, wherein the therapeutic agent comprises a Cas polypeptide and guide RNA complexed with an amphipathic molecule.

27. The SyEVs of embodiment 26, wherein the amphipathic molecule comprises a lipophilic moiety and a water-soluble polymer.

28. The SyEVs of embodiment 27, wherein the water-soluble polymer comprises poly(ethylene glycol) (PEG).

29. The SyEVs of embodiment 27 or 28, wherein the lipophilic moiety comprises a cholesterol molecule.

30. A method of making the SyEVs of any one of embodiments 1-29, the comprising:
isolating exosomes released from the eukaryotic cells;
opening the exosomes by exposing them to a mild alkaline pH;
optionally, incubating the opened exosomes with a therapeutic agent; and
closing the exosomes by applying energy to the opened exosomes, optionally wherein applying energy comprises sonication.

31. The method of embodiment 30, wherein alkaline pH is a pH of less than 10, e.g., pH8.5-9.5.

32. Extruded ghost nanovesicles (exgNVs) comprising an anti-inflammatory agent, wherein the exgNVs have an anti-inflammatory property,
enhance anti-inflammatory effect of the anti-inflammatory agent, when present, and
are produced from a human cell, and
wherein the anti-inflammatory agent is present in a therapeutically effective amount.

33. The exgNVs of embodiment 32, wherein the anti-inflammatory agent is a small molecule or a large molecule.

34. The exgNVs of embodiment 32, wherein the large molecule is a peptide or a protein.

35. The exgNVs of embodiment 32, wherein the anti-inflammatory agent is cyclosporine A.

36. The exgNVs of any one of embodiments 32-35, wherein the anti-inflammatory agent is an inhibitor of Myeloid Differentiation Primary Response 88 (Myd88).

37. The exgNVs of embodiment 36, wherein the inhibitor of MYD88 is a small molecule, e.g., T6167923 or ST2825.

38. The exgNVs of embodiment 36, wherein the inhibitor of MYD88 is a large molecule.

39. The exgNVs of embodiment 38, wherein the large molecule is a peptide.

40. The exgNVs of embodiment 39, wherein the peptide comprises the amino acid sequence RDVLPGT (SEQ ID NO:2).

41. The exgNVs of any one of embodiments 32-35, wherein the anti-inflammatory agent is an activator of the glucocorticoid receptor.

42. The exgNVs of embodiment 41, wherein the activator of the glucocorticoid receptor is a small molecule.

43. The exgNVs of any one of embodiments 32-35, wherein the anti-inflammatory agent is an inhibitor of a non-receptor tyrosine kinase.

44. The exgNVs of embodiment 43, wherein the inhibitor is Nintedanib, Imatinib, Dasatinib, gefitinib, erlotinib, or Lapatinib.

45. The exgNVs of any one of embodiments 32-35, wherein the anti-inflammatory agent is an inhibitor of Nuclear factor kappa B (NFkB) p50, AKT, extracellular-signal-regulated kinase (ERK), I kappa B kinase gamma (IKKg), Toll Like Receptor 4 (TLR4), Toll Like Receptor 2 (TLR2), Signal transducer and activator of transcription 3 (STAT3), or interleukin-1 receptor-associated kinase 4 (IRAK).

46. The exgNVs of embodiment 45, wherein the anti-inflammatory agent is a large molecule inhibitor of NFkB p50, wherein the large molecule inhibitor is a peptide comprising the amino acid sequence VQRKRQKLM.

47. The exgNVs of embodiment 45, wherein the anti-inflammatory agent is a large molecule inhibitor of AKT, wherein the large molecule inhibitor is a peptide comprising the amino acid sequence AVTDHPDRLWAWEKF (SEQ ID NO:4).

48. The exgNVs of embodiment 45, wherein the anti-inflammatory agent is a large molecule inhibitor of ERK, wherein the large molecule inhibitor is a peptide comprising the amino acid sequence MPKKKPTPIQLNP (SEQ ID NO:5).

49. The exgNVs of embodiment 45, wherein the anti-inflammatory agent is a large molecule inhibitor of IKKg, wherein the large molecule inhibitor is a peptide comprising the amino acid sequence TALDWSWLQTE (SEQ ID NO:6).

50. The exgNVs of embodiment 45, wherein the anti-inflammatory agent is a large molecule inhibitor of TLR4, wherein the large molecule inhibitor is a peptide comprising the amino acid sequence KYSFKLILAEY (SEQ ID NO:7).

51. The exgNVs of embodiment 45, wherein the anti-inflammatory agent is a large molecule inhibitor of TLR2, wherein the large molecule inhibitor is a peptide comprising the amino acid sequence PGFLRDPWCKYQML (SEQ ID NO:8).

52. The exgNVs of embodiment 45, wherein the anti-inflammatory agent is a large molecule inhibitor of STAT3, wherein the large molecule inhibitor is a peptide comprising the amino acid sequence PYLKTKAAVLLPVLLAAP (SEQ ID NO:9).

53. The exgNVs of embodiment 45, wherein the anti-inflammatory agent is a large molecule inhibitor of IRAK, wherein the large molecule inhibitor is a peptide comprising the amino acid sequence KKARFSRFAGSSPSQSSMVAR (SEQ ID NO:10).

54. The exgNVs of any one of embodiments 32-53, wherein the exgNVs are produced from a human cell line.

55. The exgNVs of any one of embodiments 32-54, wherein the human cell is a stem cell.

56. The exgNVs of embodiment 55, wherein the stem cell is a mesenchymal stem cell.

57. The exgNVs of any one of embodiments 32-56, wherein the exgNVs are prepared by a process that comprises disruption of a human cell followed by exposure to high pH prior to forming the exgNVs and wherein the exgNVs comprise an enrichment of mitochondrial proteins as compared to vesicles made from the same type of cell by exposure to high pH in absence of the disruption step, wherein the disruption of a human cell optionally comprises applying energy to the cell, e.g., by sonication or comprises serial extrusion.

58. The exgNVs of any one of embodiments 32-56, wherein the exgNVs are prepared by a process that comprises disruption of a human cell followed by exposure to high pH prior to forming the exgNVs and wherein the exgNVs comprise a reduced amount of one or more of cytoplasmic proteins, lysosomal proteins, exosomal proteins, plasma membrane proteins, and endoplasmic reticulum proteins as compared to vesicles made from the same type of cell by exposure to high pH in absence of the disruption step, wherein the disruption of a human cell optionally comprises applying energy to the cell, e.g., by sonication or comprises serial extrusion.

59. The exgNVs of any one of embodiments 32-56, wherein the exgNVs are prepared by a process that comprises disruption of a human cell followed by exposure to high pH prior to forming the gNVs and wherein the gNVs comprise an enrichment of mitochondrial proteins and a reduced amount of cytoplasmic proteins as compared to vesicles made from the same type of cell by exposure to high pH in absence of the disruption step, wherein the disruption of a human cell optionally comprises applying energy to the cell, e.g., by sonication or comprises serial extrusion.

60. The exgNVs of any one of embodiments 32-56, wherein the exgNVs are prepared by a process that comprises disruption of a human cell followed by exposure to high pH prior to forming the exgNVs and wherein the exgNVs reduce TNF-α production while vesicles made from the same type of cell by exposure to high pH in absence of the disruption step do not significantly reduce TNF-α production, wherein the disruption of a human cell optionally comprises applying energy to the cell, e.g., by sonication or comprises serial extrusion.

61. The exgNVs of any one of embodiments 57-60, wherein the high pH comprises an alkaline pH of 7.5-13.5.

62. The exgNVs of embodiment 61, wherein the alkaline pH is a pH of less than 10, e.g., pH8-9.5.

63. The exgNVs of embodiment 63, wherein the alkaline pH is pH 9.

64. The exgNVs of any one of embodiments 32-63, wherein the anti-inflammatory property of the gNVs comprise reduction of production of a pro-inflammatory cytokine.

65. The exgNVs of embodiment 64, wherein the pro-inflammatory cytokine comprises IL-2, IL-6, IL-12, or TNF-α.

66. The exgNVs of any one of embodiments 32-65, wherein the anti-inflammatory agent reduces IL-6 secretion.

67. The exgNVs of any one of embodiments 32-66, wherein the anti-inflammatory agent reduces IL-2 secretion.

68. The exgNVs of any one of embodiments 32-67, wherein the anti-inflammatory agent reduces IL-12 secretion.

69. The exgNVs of any one of embodiments 32-68 for use in a method for treating an inflammatory condition in a subject.

70. A method for treating an inflammatory condition in a subject, the method comprising:
administering a therapeutically effective amount of the SyEVs of any one of embodiments 1-31 or the exgNVs of any one of embodiments 32-69 to the subject.

71. The exgNVs for use of embodiment 69 or the method of embodiment 70, wherein the subject has cancer, multiple sclerosis, psoriasis, dry eye disease, asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory condition, demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis, stroke, or ankylosing spondylitis.

72. The method of embodiment 70 or 71, wherein the administering comprises intravenous administration.

73. The method of embodiment 70 or 71, wherein the administering comprises subcutaneous administration.

74. The method of embodiment 70 or 71, wherein the administering comprises intramuscular, intraperitoneal, intraarterial, intraarticular, intracerebral (intraparenchymal) or intracerebroventricular administration.

75. The method of embodiment 70 or 71, wherein the administering comprises local administration to a target site in the subject.

76. The method of embodiment 75, wherein the target site comprises or is susceptible to developing an inflammatory response.

77. The method of embodiment 75 or 76, wherein the target site has an injury.

78. The method of embodiment 75 or 76, wherein the target site is adjacent to a site that has injury.

79. The method of embodiment 75-78, wherein the target site comprises a site in the central nervous system.

80. The method of embodiment 79, wherein the target site comprises brain.

81. The method of embodiment 80, wherein the target site comprises site of arterial blockage or hemorrhage.

82. The method of embodiment 81, wherein the administering is intraarterial administering.

83. Extruded ghost nanovesicles (exgNVs) comprising a Cas polypeptide and guide RNA complexed with an amphipathic molecule.

84. The exgNVs of embodiment 83, wherein the amphipathic molecule comprises a lipophilic moiety and a water-soluble polymer.

85. The exgNVs of embodiment 84, wherein the water-soluble polymer comprises poly(ethylene glycol) (PEG).

86. The exgNVs of embodiment 84 or 85, wherein the lipophilic moiety comprises a cholesterol molecule.

87. A method of making the exgNVs of any one of embodiments 83-86, the method comprising:
serially extruding cells, such as, mammalian cells to generate vesicles;
opening the vesicles by exposing them to an alkaline pH;
incubating opened vesicles with a Cas polypeptide and guide RNA complexed with an amphipathic molecule; and
closing the vesicles by applying energy to the vesicles, optionally wherein applying energy comprises sonication.

88. The method of embodiment 87, wherein alkaline pH is a pH of less than 10, e.g., pH8.5-9.5.

89. A method of delivering a Cas polypeptide and guide RNA to a cell, the method comprising:
contacting the cell with the exgNVs of any one of embodiments 83-86.

90. Nanovesicles (NVs) produced from a human cell line genetically modified to express lymphocyte function-associated antigen-1 (LFA-1) or macrophage-1 antigen (Mac-1), wherein LFA-1 or Mac-1 is located on surface of the NVs.

91. The NVs of embodiment 90, wherein the NVs are produced from the human cell line by serial extrusion.

92. The NVs of embodiment 91, wherein the NVs are extruded ghost nanovesicles (exgNVs).

93. The NVs of embodiment 90, wherein the NVs are secreted from the human cell line.

94. The NVs of embodiment 93, wherein the NVs are SyEVs prepared from the NVs secreted from the human cell line.

95. The NVs of any one of embodiments 90-94, wherein NVs are loaded with a therapeutic agent.

96. The NVs of embodiment 95, wherein the therapeutic agent is a small molecule or a large molecule.

97. The NVs of embodiment 95, wherein the therapeutic agent comprises:
an anti-inflammatory agent; or
a Cas polypeptide and guide RNA complexed with an amphipathic molecule.

98. The NVs of embodiment 97, wherein the amphipathic molecule comprises a lipophilic moiety and a water-soluble polymer.

99. The NVs of embodiment 98, wherein the water-soluble polymer comprises poly(ethylene glycol) (PEG).

100. The exgNVs of embodiment 98 or 99, wherein the lipophilic moiety comprises a cholesterol molecule.

101. A method of making the exgNVs from a human cell line genetically modified to express lymphocyte function-associated antigen-1 (LFA-1) or macrophage-1 antigen (Mac-1), the method comprising:
serially extruding the human cell line to generate vesicles;
opening the vesicles by exposing them to an alkaline pH;
incubating opened vesicles with a therapeutic agent; and
closing the vesicles by applying energy to the cell, optionally wherein applying energy comprises sonication.

102. A method of making the SyEVs from a human cell line genetically modified to express lymphocyte function-associated antigen-1 (LFA-1) or macrophage-1 antigen (Mac-1), the method comprising:
isolating exosomes released from the human cell line;
opening the exosomes by exposing them to an alkaline pH;
optionally, incubating the opened exosomes with a therapeutic agent; and
closing the exosomes by applying energy to the opened exosomes, optionally wherein applying energy comprises sonication.

103. The method of embodiment 101 or 102, wherein alkaline pH is a pH of less than 10, e.g., pH8.5-9.5.

104. A method for delivering a therapeutic agent to a cell expressing a receptor of LFA-1 and/or Mac-1, the method comprising contacting the cell with the NVs of any one of embodiments 90-100.

105. The method of embodiment 104, wherein the receptor is a receptor for LFA-1 and Mac-1.

106. The method of embodiment 105, wherein the receptor is Intercellular Adhesion Molecule 1 (ICAM-1).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: ExgNVs Loaded with an Anti-Inflammatory Agent

### Methods

Preparation of exgNVs and Loading of Anti-Inflammatory Agent

MSCs were resuspended at a density of 5×10⁶ cells per mL in total 10 mL of phosphate buffered saline. Cell suspensions were passed five times through each of the membrane filters with a pore size of 10 μm, 5 μm and 1 μm, in that order. Respectively 1 and 2 mL of 50 and 10% solution of iodixanol (Axis-Shield PoC AS), followed by 7 mL of the cell suspension effluent from the membrane filter were sequentially added to each 10 mL ultracentrifuge tube. The layers formed between 50% iodixanol and 10% iodixanol after ultracentrifugation at 100,000×g for 2 hours were collected and considered NVs. The NVs were incubated with high pH solution (200 mM Na₂CO₃, pH 14.0) for 1 hour at 25 degree. The solution was applied to 4 mL of 50% iodixanol, followed by addition of 4 mL of 30% iodixanol and 2 mL of 10% iodixanol to ultracentrifuge tube. The layers formed between 10% and 30% iodixanol after ultracentrifugation at 100,000×g for 2 hours was collected. Finally, the samples were sonicated for 30 min, and considered NVs (FIG. 1A).

For generating exgNVs loaded with an anti-inflammatory agent, the layers formed between 10% and 30% iodixanol after ultracentrifugation at 100,000×g for 2 hours was collected and sonicated with an anti-inflammatory agent for 30 min to generate exgNVs loaded with the agent. After sonication, the exgNVs were separated from free anti-inflammatory agent by ultracentrifugation. See FIG. 1B. The following small molecule agents were loaded: T6167923 or Cyclosporin (CsA). The following large molecule agents were loaded: anti-Myd88 peptide: RDVLPGT (SEQ ID NO:2) “Peptide” or siRNA (Sense: 5′ UAAGGCUAUGAAGAGAUACUU 3′ (SEQ ID NO:11); Antisense: 5′ GUAUCUCUUCAUAGCCUUAUU 3′ (SEQ ID NO:12)).

### LPS-Induced Inflammatory Response

Natural LPS (100 ng/mL) was added to the RAW 264.7 cells for 3 hours, followed by treatment of T6167923 (50 μM), exgNVs or exgNV^{T6167923} (5×10⁸) for 15 hours.

For in vivo study, mice (wild-type mice of the C57BL/6 genetic background, 6 weeks old) were intraperitoneally injected with E. coli OMVs to induce systemic inflammation, followed by intraperitoneal injection of exgNVs, exgNV^{Peptide} or exgNV^{T6167923} (2×10⁹) after one hour. Mice were sacrificed at 6 hours following anesthetization with intraperitoneal injection of xylazine chloride (Bayer) and ketamine hydrochloride (Pfizer). Blood and BAL fluid were collected from mice, and then cytokines in the supernatant were analyzed by DuoSet ELISA Development kit (R&D Systems).

### PMA/Calcium Ionomycin-Induced

PMA (10 nM) and calcium ionomycin (1 μM) were added to the T jurkat cells for 24 hours, together with CsA (0.5 ng), exgNVs or exgNV^{CsA} (10⁵).

Loading of Cas9 Protein into exgNVs

Cas9 protein, exgNVs and exgNV^{Cas9} were separated by 10% SDS-PAGE and the separated gel was transferred to a polyvinylidene difluoride membrane. The blocked membrane was then incubated with anti-Cas9 antibody (Thermo Fisher Scientific) to confirm the delivery of Cas9. After incubation with horseradish peroxidase-conjugated secondary antibody, the immunoreactive bands were visualized with a chemiluminescent substrate.

### Results

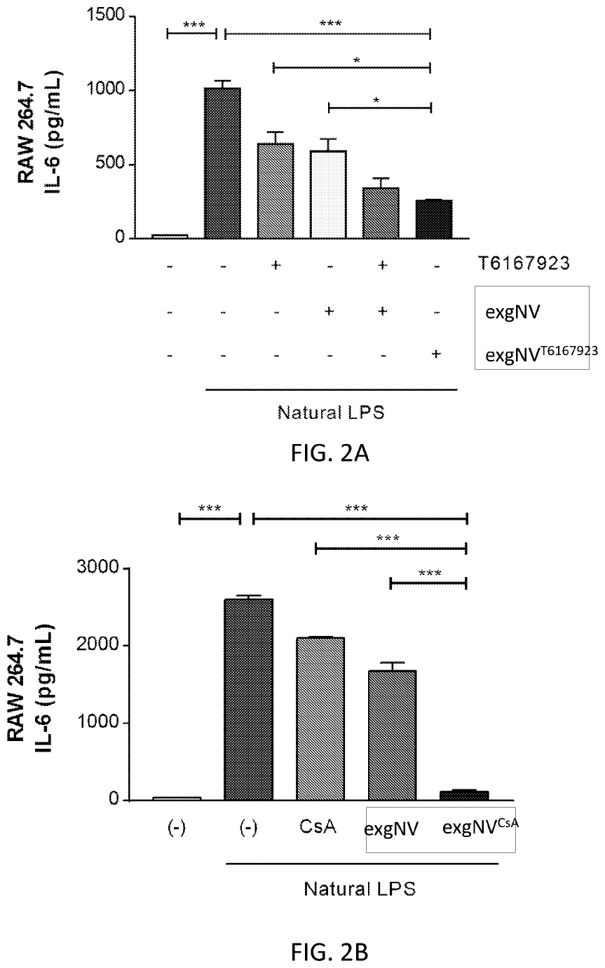

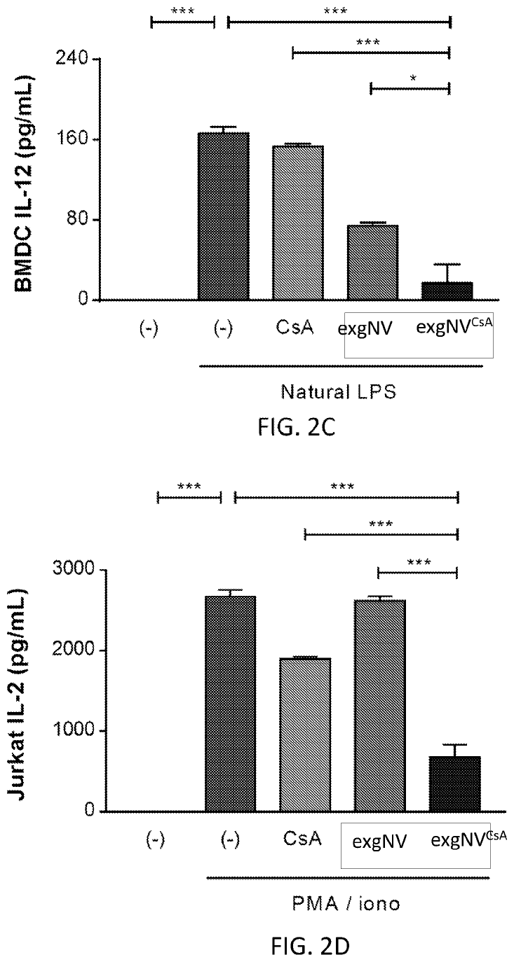

FIG. 2A is a graph showing reduction in natural LPS-induced IL-6 by ExgNVs loaded with T6167923 (small molecule inhibitor of Myd88). Natural LPS (100 ng/mL) was added to the RAW 264.7 cells for 3 hours, followed by treatment of T6167923 (50 μM), ExgNVs or ExgNV^{T6167923} (5×10⁸) for 15 hours. Supernatant concentration of IL-6 was measured by ELISA. *, P<0.05; ***, P<0.001; one way ANOVA with Tukey's multiple comparison test. Error bars indicate SEM. N=2. Treatment with exgNV^{T6167923} resulted in a higher than expected effect on reduction of pro-inflammatory cytokine, IL-6 as compared to the effect of exgNVs or T6167923 alone.

FIG. 2B is a graph showing reduction in natural LPS-induced IL-6 by ExgNVs loaded with cyclosporine A (CsA). Natural LPS (100 ng/mL) was added to the RAW 264.7 cells for 3 hours, followed by treatment of CsA (5 μg), ExgNVs or ExgNV^{CsA} (10⁹) for 15 hours. Supernatant concentration of IL-6 was measured by ELISA. ***, P<0.001; one way ANOVA with Tukey's multiple comparison test. Error bars indicate SEM. N=2. Treatment with exgNV^{CsA} resulted in a higher than expected effect on reduction of pro-inflammatory cytokine, IL-6 as compared to the effect of exgNVs or CsA alone.

FIG. 2C is a graph showing reduction in natural LPS-induced IL-12 by ExgNVs loaded with CsA. Natural LPS (100 ng/mL) was added to the mouse bone marrow-derived dendritic cells for 3 hours, followed by treatment of CsA (5 μg), ExgNVs or ExgNV^{CsA} (10⁹) for 15 hours. Supernatant concentration of IL-12 was measured by ELISA. *, P<0.05; ***, P<0.001; one way ANOVA with Tukey's multiple comparison test. Error bars indicate SEM. N=2. Treatment with exgNV^{CsA} resulted in a higher than expected effect on reduction of pro-inflammatory cytokine, IL-12 as compared to the effect of exgNVs or CsA alone.

FIG. 2D is a graph showing reduction in PMA/calcium ionomycin-induced IL-2 by ExgNVs loaded with CsA. PMA (10 nM) and calcium ionomycin (1 μM) were added to the T jurkat cells for 24 hours, together with CsA (0.5 ng), ExgNVs or ExgNV^{CsA} (10⁵). Supernatant concentration of IL-2 was measured by ELISA. ***, P<0.001; one way ANOVA with Tukey's multiple comparison test. Error bars indicate SEM. N=2. Treatment with exgNV^{CsA} resulted in a higher than expected effect on reduction of pro-inflammatory cytokine, IL-2 as compared to the effect of exgNVs or CsA alone.

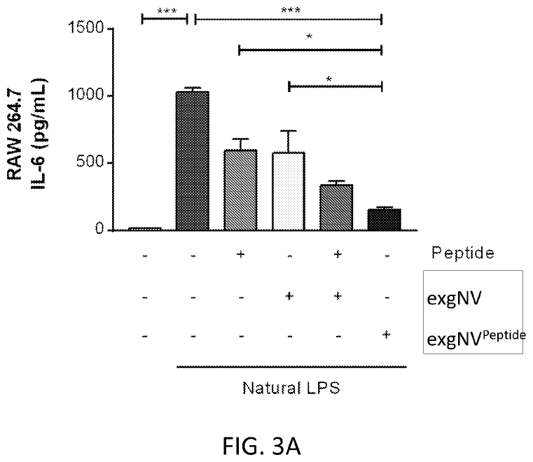

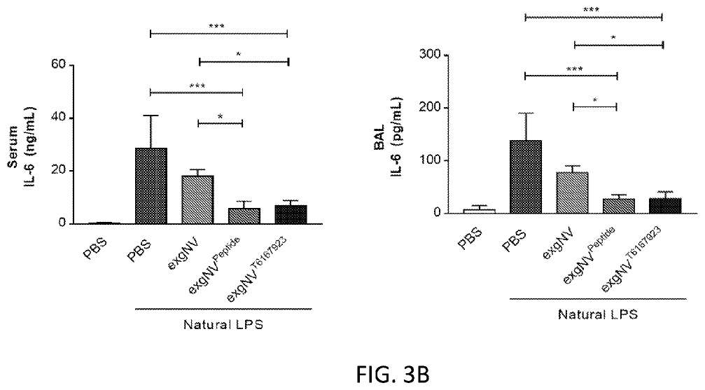

FIG. 3A is a graph showing reduction in natural LPS-induced IL-6 by ExgNVs loaded with anti-Myd88 peptide. Natural LPS (100 ng/mL) was added to the RAW 264.7 cells for 3 hours, followed by treatment of peptide (0.5 μg), ExgNVs or ExgNV^{Peptide} (5×10⁸) for 15 hours. Supernatant concentration of IL-6 was measured by ELISA. *, P<0.05; ***, P<0.001; one way ANOVA with Tukey's multiple comparison test. Error bars indicate SEM. N=2. Treatment with exgNV Peptide resulted in a higher than expected effect on reduction of pro-inflammatory cytokine, IL-6 as compared to the effect of exgNVs or peptide alone.

FIG. 3B shows inflammatory cytokines IL-6 in the serum (left) and BAL fluid (right) at 6 h of mice injected intraperitoneally with OMVs, followed by intraperitoneal injection of exgNVs, exgNV^{Peptide} or exgNV^{T6167923} (2×10⁹). *, P<0.05; ***, P<0.001; one way ANOVA with Tukey's multiple comparison test. Error bars indicate SEM. N=5. Administration of exgNVs loaded with anti-inflammatory agents such as T6167923 or peptides reduced OMV-induced IL-6 production more effectively than empty exgNVs.

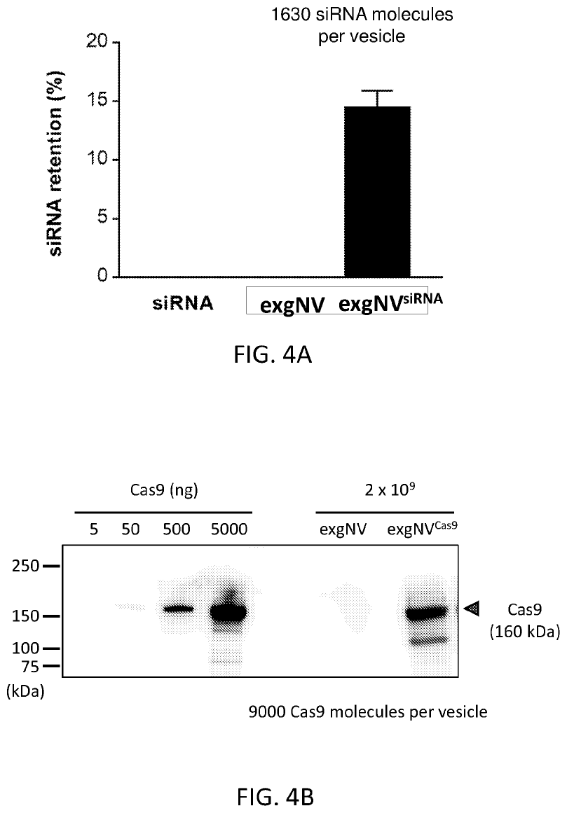

FIG. 4A shows siRNA retention in the layer between 10% and 30% iodixanol solution following density gradient ultracentrifugation of siRNA, ExgNVs or ExgNV^{siRNA}.

FIG. 4B shows Western blot analysis of Cas9 protein, exgNVs and exgNVs loaded with Cas9 (exgNV^{Cas9}) with anti-Cas9 antibody. exgNVs could be efficiently associated with large size of Cas9 (9000 Cas9 molecules per vesicle), revealing that exgNV platform might be used for gene editing.

### Example 2: Comparison of ExgNVs to gNVs

Preparation of gNVs

gNVs were prepared as described in Adv Health Mater. 2019 February; 8(4):e1801082 and WO2016133254. Briefly, cells were exposed to an alkaline solution (200 mM Na₂CO₃, 1× phosphatase inhibitor, pH 11.5), and then sonicated, followed by ultracentrifuged (100,000×g for 15 mins) to get membrane sheets. The membrane pellets were resuspended with PBS and subjected to sonication. The final vesicles were collected between 10% and 30% iodixanol after ultracentrifugation at 100,000×g for 2 hours.

### Preparation of OMVs

E. coli cultures were pelleted at 6,000×g, 4° C. for 20 min, twice, and then the supernatant fraction was filtered through a 0.45-μm vacuum filter and was concentrated by ultrafiltration Vivaflow 200 module (Sartorius) with a 100 kDa cut-off membrane. The retentate was again filtered through a 0.22-μm vacuum filter to remove any remaining cells. The resulting filtrate was subjected to ultracentrifugation at 150,000×g, 4° C. for 3 h and resuspended in PBS.

### RNA and DNA Analysis

RNA from gNVs and exgNVs was isolated using miRCURY™ RNA isolation kit for biofluids (Exiqon) according to manufacturer's protocol. DNA was isolated using Qiamp DNA Blood Mini kit (Qiagen) according to manufacturer's protocol. One microliter of isolated RNA or DNA were analyzed for its quality, yield, and nucleotide length with capillary electrophoresis using Agilent RNA 6000 Nanochip and Agilent High sensitivity DNA chip, respectively, on an Agilent 2100 Bioanalyzer® (Agilent Technologies).

### LC-MS/MS Analysis

exgNVs and gNVs were digested with trypsin using the filter-aided sample preparation (FASP) method and C18 spin columns desalting according to manufacturer's instructions. All fractions were dried on Speedvac and reconstituted in 3% acetonitrile and 0.2% formic acid and analyzed on Orbitrap Fusion Tribrid mass spectrometer interfaced with Easy-nLC 1200 (Thermo Fisher Scientific, Waltham, MA). Peptides were trapped on the Acclaim Pepmap 100 C18 trap column (100 μm×2 cm, particle size 5 μm; Thermo Fischer Scientific) and separated on the in-house packed C18 analytical column (75 μm×30 cm, particle size 3 μm) using the gradient from 5% to 33% B in 160 min, from 33% to 100% B in 5 min, solvent A was 0.2% formic acid and solvent B was 80% acetonitrile and 0.2% formic acid. Precursor ion mass spectra were recorded at 120 000 resolution, the most intense precursor ions were selected, fragmented using HCD at collision energy setting of 30 and the MS/MS spectra were recorded at 30 000 resolution with the maximum injection time of 125 ms and the isolation window of 1.0 Da. Charge states 2 to 7 were selected for fragmentation, dynamic exclusion was set to 45 s with 10 ppm tolerance.

Effect of gNVs and exgNVs on OMV-Induced Inflammation

RAW 264.7 (1×10⁵), a mouse macrophage cell line, were seeded into 24-well plates, and then E. coli OMVs (100 ng/mL) were treated for 3 hours to induce inflammation. exgNVs and gNVs (1×10⁹) were applied to the cells, and the supernatant concentrations of TNF-α and IL-6 at 15 hours later were measured by ELISA kit (R&D systems).

### Results

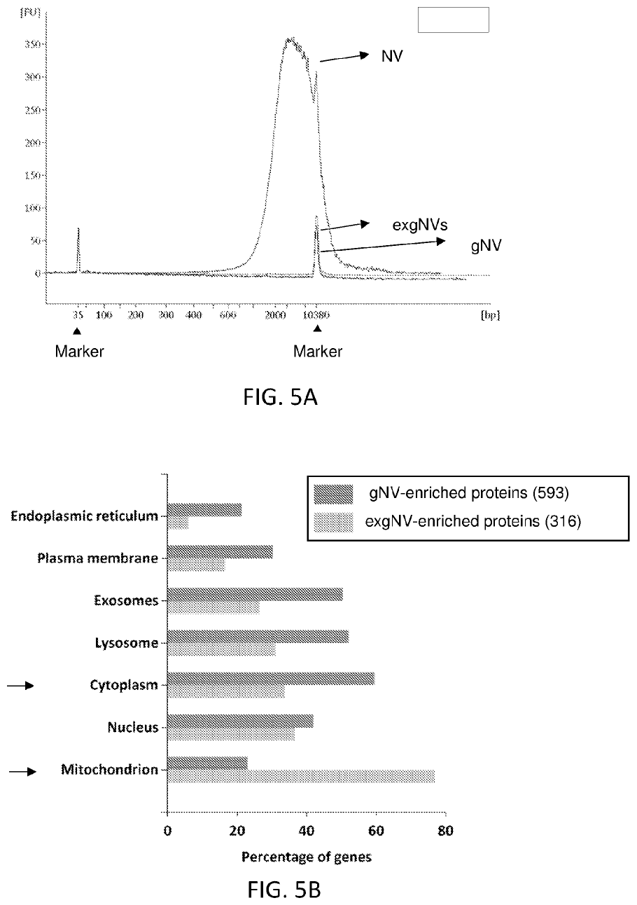

FIG. 5A shows that both exgNVs and gNVs have minimal DNA content. In comparison, NVs have substantial amount of DNA.

593 proteins were identified from gNVs, while 316 proteins were identified from exgNVs. In the GO term subcellular localization analysis, gNV-enriched proteome showed distinct feature from exgNV-enriched proteome (FIG. 5B). gNV proteome was enriched cytoplasmic proteins, whereas gNV proteome was enriched for mitochondrial proteins. Example 3: gNVs reduce OMV-induced inflammation

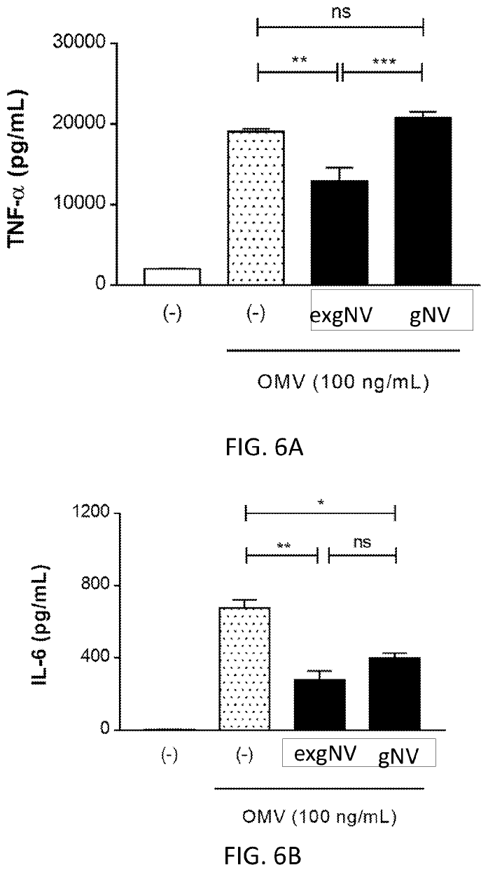

FIG. 6A shows that there is a difference between the effect of gNVs and exgNVs on level of TNF-α.

FIG. 6B shows that gNVs and exgNVs have a similar effect on level of IL-6.

### Example 3: Generation of Cell Lines Expressing LFA-1 HEK 293 Cell Line

The cells transfected in this study are a derivate of the HEK 293 cell line called FreeStyle™ 293-F Cells (293-F). These cells have been adapted to grow in suspension in FreeStyle™ 293 Expression Medium (Thermo Fisher).

### Jurkat Cell Line

The Jurkat cells used in this study was sent to be profiled by the ATCC™ cell line authentication service. The analysis showed that these cells were like the ATCC human cell line CRL-2572. This cell line is a FADD mutant of the Jurkat cell line.

### Generation of Transformed Cells

In this study a stable clone of the 293-F cell line was developed to be used for the generation of NVs. Thus, the study was comprised of culturing 293-F cells, transfecting them and selecting a clone for the best expression. The expression of the membrane proteins and the functionality of the receptor was analysed via multiple methods. On cells growing in suspension the FectoPro™ system (Polyplus) was used for transfection and on cells growing adherently the Lipofectamine™ 2000 (Thermo Fisher) system was used. The negative control for the transfections were called Mock and goes through the same protocol as the cells that are transfected but without DNA.

Two plasmids were designed from the same cloning vector called pcDNA3.1(+) (GenScript) but with different versions. One of them was customized to contain the gene encoding CD11a and resistance for hygromycin B (Hygro B) in pcDNA3.1/Hygro(+). The other plasmid was customized to contain the gene encoding CD18 in pcDNA3.1(+). The standard cloning vector pcDNA3.1(+) has the selection marker NeoR which is a gene that provides resistance for neomycin. Although the antibiotic used after the transfection with the CD18 plasmid was geneticin (G418) which is an analogue for neomycin and works in a similar way.

The transfections of cells growing in suspension were performed with the FectoPro™ system according to the manufacturer's (Polyplus) protocol. Briefly, the day prior to the transfection the 293-F cells were split to a pre-culture with the concentration of 1*10⁶ cells/mL. On the day of transfection, the cells were split to a concentration of 2*10⁶ cells/mL and moved to the appropriate vessel with the intended volume of Freestyle™ 293. IMDM medium (Lonza) was used as a dilution medium and no Fecto Pro booster was used. The dilution medium, 0.1 mL/mL final volume, was added to a sterile tube with the plasmid (see Method 3.2.2), 0.8 μg/mL final volume, and the transfection reagent (Polyplus), 0.8 μL/mL final volume. To make the Mock the same mixture was made but instead of adding the plasmid, more IMDM medium was added. The mixture was incubated for 10-30 minutes at room temperature and then added to the cells to make the final volume and mixed. The cells were then put in the incubator at 37° C.

The transfections of cells growing adherently were performed with the Lipofectamine™ 2000 system according to the manufacturer's (Thermo Fisher) protocol. Briefly, two days prior to the transfection the cells to be transfected was seeded out on a 6-well plate, according to FIG. 3 in 2 mL growth medium (Freestyle™ 293 medium with 10% FBS).

After each transfection there was a pool of clones, both transfected and non-transfected. Therefore 48 hours after transfection the medium was changed, and antibiotics were added with the new medium. Thus, the non-transfected cells became non-viable due to the antibiotics.

Clones were started to be picked after 1-3 weeks and the cells in the Mock were non-viable. Between 30-50 clones in total were picked and expanded and then the expression of the membrane proteins were tested.

To determine whether the transfections were successful and hence if the membrane proteins were expressed on the membrane, 100,000 transfected cells were analysed with flow cytometry. The flow cytometer used was either a BD FACS Aria II Cell sorter or a BD FACSVerse™ flow Cytometry running BD FACSSuite™ software (BD Biosciences).

### Extrusion of Cells

Via a serial extrusion high quantity of NVs can be made from different types of cells. A mini-extruder (Avanti Polar Lipids) was utilized with filters and a polycarbonate membrane filter (Whatman) with a pore size that diminishes between each extrusion in the series. In this project both Jurkat cells and different clones of 293-F cells was processed accordingly and the NVs was then purified via ultra-centrifugation. The presence of CD11a and CD18 on the NV were then tested by nano-FCM to determine whether the NVs kept the expression of membrane receptors.

The cells that were used for the extrusion were washed once with PBS and then resuspended in a concentration of 2-5*10⁶ cells/mL. The cells were then extruded three times through membranes with a diminishing pore size of 10, 5 and 1 μm. The NVs were then purified via ultracentrifugation in a two-step gradient consisting of 2 mL 10% and 1 mL 50% iodixanol. The 50% layer was at the bottom of an ultracentrifuge tube and the 10% were carefully pipetted on top of the 50% layer and then the sample was added on top of the 10% layer. The tube was then ultracentrifuged for 2 hours at 100,000 g at 4° C. A layer between the 10% and 50% iodixanol was the collected and analyzed.

### Nano-FCM

8 μL of the NVs was incubated for 40 minutes with 1 μL of each of the antibodies (anti-CD11a and anti-CD18), total sample volume was 10 μL. The NVs were then washed with 2.5 mL PBS by ultracentrifugation. This ultracentrifugation was for 30 minutes, at 52,000 rpm and 4° C. The NVs were then resuspended in 50 μL PBS and analysed with the nano-FCM. Only PBS was measured for background as comparison and then gated in the analysis alongside the populations of NVs.

100,000 293f cells transfected with CD18 were incubated in 50 μL human IgG (1 mg/mL in D-PBS) for 15 minutes at 4° C. Antibodies specific for CD18 (APC) and CD11a (PE) was added together with a viability dye to the cells and then incubated for 30 minutes at 4° C. In the analysis 10,000 events were collected and collected data were analysed. Gating for the cells that were negative for the viability dye was used so that only the viable cells expressions of the two membrane proteins were analysed.

100,000 CD18 positive cells transfected with CD11a were incubated in 50 μL human IgG (1 mg/mL in D-PBS) for 15 minutes at 4° C. Antibodies specific for CD18 (APC) and CD11a (PE) was added together with a viability dye to the cells and then incubated for 30 minutes at 4° C. In the analysis 10,000 events were collected and collected data were analysed. Gating for the cells that were negative for the viability dye was used so that only the viable cells expressions of the two membrane proteins were analysed.

### Western Blot

NVs prepared from cells expressing LFA-1, a CD18 clone, Jurkat cells and wildtype 293f cells were separated on NuPAGE™ 4-12% Bis-Tris gels. After blocking the membranes, one membrane was incubated with an anti-CD11a antibody and the other one with anti-CD18 antibody. After incubation, the secondary antibodies used were labelled with alkaline phosphatase (AP) and the membrane was developed using 5 mL BCIP/NBT one component membrane substrate.

### Cell Binding Assay

A 96-well plate were coated with the ICAM-1, by adding 100 μL of a solution with a concentration of 5 μg/mL. The plate was left overnight at 4° C. and then blocked using a 1% BSA solution and incubated at room temperature. 50 000 labelled cells were then added to each well, the cells were either an LFA-1 expressing clone or non-transfected 293f cells. The plate was incubated at 37° C. for 1 hour, washed and analysed.

### Results

Double transfection of 293F cells failed to generate viable cells. An alternative strategy to generate double transfectants was pursued. Cell lines transfected with plasmid encoding CD18 were generated. These cells where then transfected with plasmid encoding CD11a, thereby generating cells that co-express CD11a and CD18.

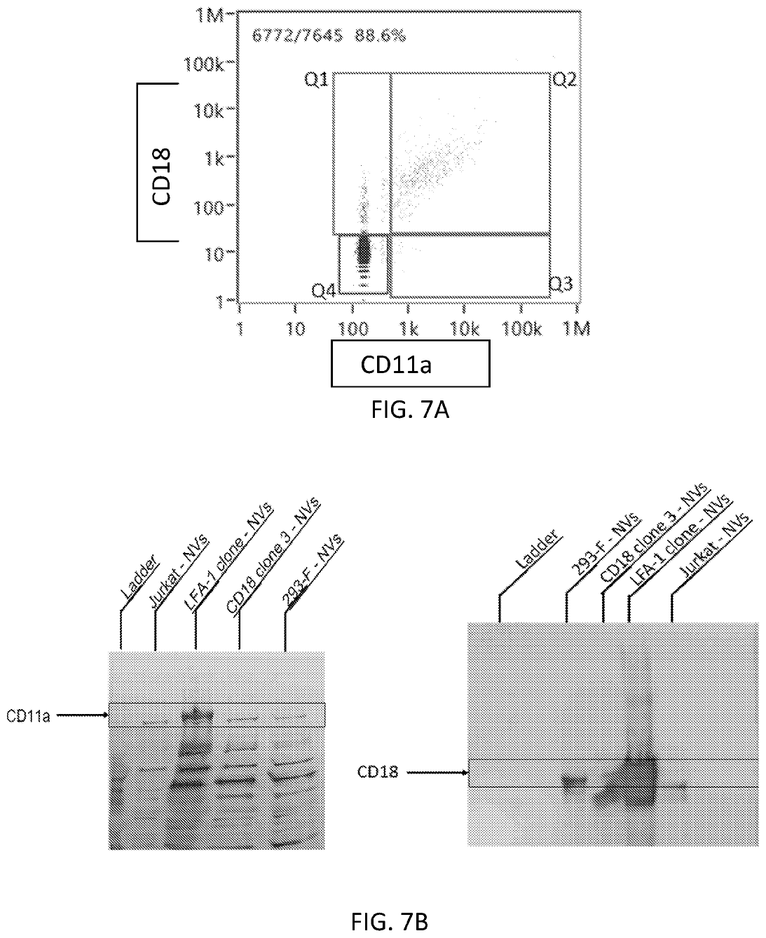

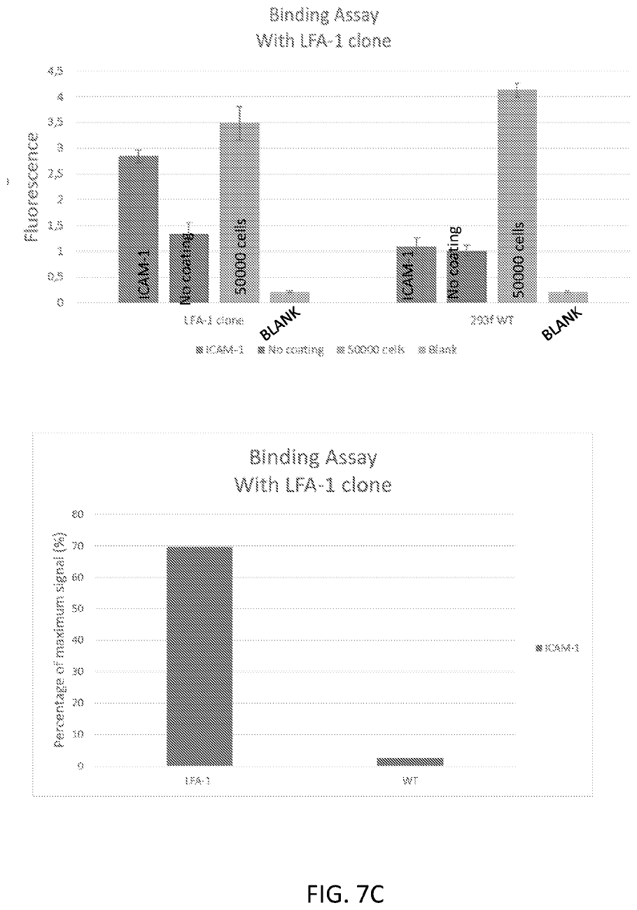

FIG. 7A shows Nano-FCM data on NVs prepared from 293f cells expressing LFA-1. The NVs analysed had a big double positive population in Q2. This indicates that CD11a and CD18 were present on the surface of the NVs prepared from LFA-1 positive 293f cells.

FIG. 7B shows the Western blot analysis of NVs prepared from an LFA-1 clone, a CD18 clone, 293f cells and Jurkat cells incubated with either anti-CD11a antibody (left) and anti-CD18 antibody (right). The NVs analyzed had a strong band for both CD11a and CD18 indicating that the expression of LFA-1 was maintained on the NVs.

FIG. 7C (top) shows a binding assay comparing cells expressing LFA-1 and non-transfected 293f cells. The assay showed a higher signal from the wells coated than the non-coated wells indicates that LFA-1 can successfully bind to ICAM-1, thus a functional LFA-1 is expressed on the cells. FIG. 7C (bottom) shows binding of cells expressing LFA-1 and non-transfected 293f cells to wells coated with ICAM-1 as percent binding with background subtracted.

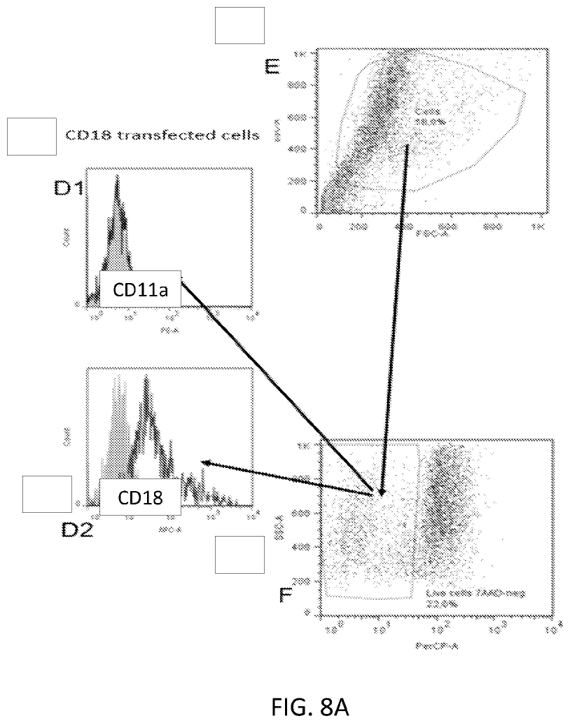

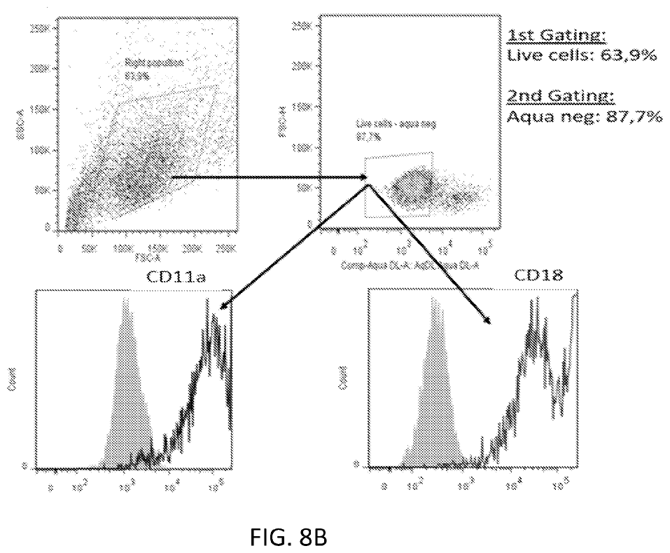

FIG. 8A shows the flow cytometry analysis of the cells transfected with CD18. From the gating steps only viable cells were analysed for the their expression of CD18. After the analysis there is a clear population of CD18 positive cells.

FIG. 8B shows the flow cytometry analysis of the cells transfected with CD11a. From the gating steps only viable cells were analysed for the their expression of CD11a. After the analysis there is a clear population of CD11a positive cells.

### Example 4: Generation of SyEV Loaded with Cas9/CRISPR

### Methods

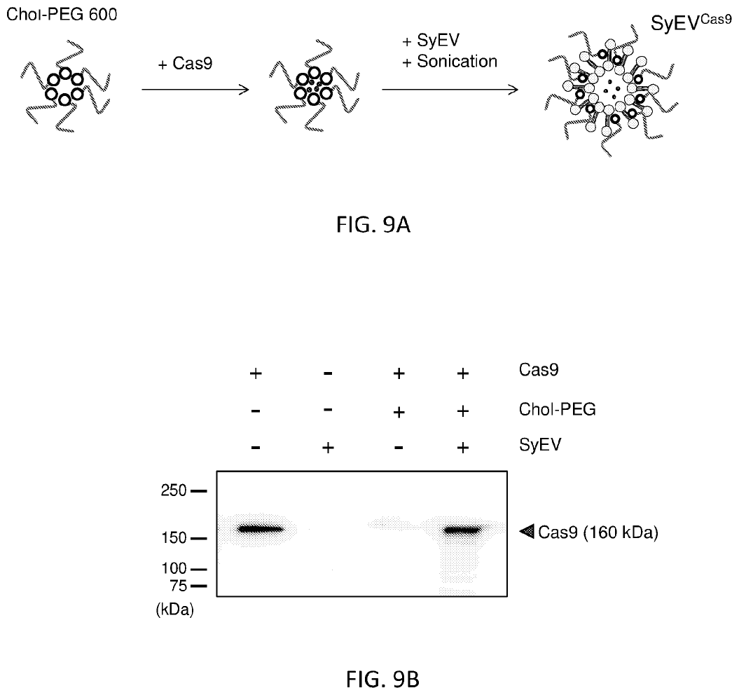

Loading of Cas9 into SyEV: Cholesterol-PEG 600 (Sigma Aldrich) was dissolved in water to make 2 mg/mL, and then added with 50 μg of Cas9 (IDT Technologies), followed by further incubation overnight at room temperature. MSC-derived membranes were isolated by treatment with high pH solution (200 mM Na₂CO₃, pH 11), and sonicated with Cholestero-PEG 600-Cas9 complex as produced above. The encapsulated SyEV (SyEV^{Cas9}) were separated from free Cholestero-PEG 600-Cas9 after ultracentrifugation at 100,000×g for 2 hours (FIG. 9A).

Western blot: Cholesterol-PEG 600, Cas9, SyEV and different combinations for these compounds were separated by 10% SDS-PAGE and the separated gel was transferred to a polyvinylidene difluoride membrane. The blocked membrane was then incubated with anti-Cas9 antibody (Thermo Fisher Scientific) to confirm the delivery of Cas9. After incubation with horseradish peroxidase-conjugated secondary antibody, the immunoreactive bands were visualized with a chemiluminescent substrate.

### Results

Cas9 protein was successfully incorporated into SyEV with the help of Cholesterol-PEG, and interestingly, SyEV were essential for packaging of Cas9 (FIG. 9B).

### Example 5: SyEV-Mediated Gene Editing in GFP-Overexpressing Cell Lines

### Methods

Loading of CRISPR complex into SyEV: Cholesterol-PEG 600 (Sigma Aldrich) was dissolved in water, and then incubated with Cas9 and specific guide RNA against GFP gene (IDT Technologies) overnight. MSC-derived membranes were isolated by treatment with high pH solution (200 mM Na₂CO₃, pH 11), and sonicated with CRISPR complex as produced above. The loaded SyEV^{CRISPR} were separated from free complex after ultracentrifugation at 100,000×g for 2 hours (FIG. 10A).

Flow cytometry: The GFP-overexpressing cells were incubated with SyEV^{CRISPR} (10¹⁰) or lipofectamine for 48 hours, and then the cells were firstly stained with 7-Aminoactinomycin D (Sigma Aldrich) to exclude dead cells. Events were collected and analyzed by using a Fortessa-X20 Flow cytometer (BD Biosciences) and FlowJo software (Tree Star).

### Results

MSC-SyEV loaded with Cas9 and guide RNA against GFP could efficiently inhibit the GFP expression (30%) on target cells (FIG. 10B). However, the loaded HEK293-SyEV showed relatively weak response for the inhibition of GFP fluorescence, indicating that the efficiency of targeted drug delivery is different depending on the cell source of vesicles.

### Example 6: Engineering of EV to Overexpress LFA-1 on their Surface

### Methods

EV isolation: The cell supernatants were centrifuged at 300×g for 10 min and 2,000×g for 20 min at 4° C. to remove cell debris. The resulting supernatants were ultracentrifuged at 16,500×g for 20 min and 120,000×g for 2.5 h at 4° C. to collect large and small exosomes, respectively. The large and small exosomes were pooled together for further experiment.

NVs generation: The cells that were used for the extrusion were washed once with PBS and then resuspended in a concentration of 2-5*106 cells/mL. The cells were then extruded three times through membranes with a diminishing pore size of 10, 5 and 1 μm. The NVs were then purified via ultracentrifugation in a two-step gradient consisting of 2 mL 10% and 1 mL 50% iodixanol. The 50% layer was at the bottom of an ultracentrifuge tube and the 10% were carefully pipetted on top of the 50% layer and then the sample was added on top of the 10% layer. The tube was then ultracentrifuged for 2 hours at 100,000 g at 4° C. A layer between the 10% and 50% iodixanol contains NVs which was collected and analyzed.

Nano-FCM analysis: Different vesicle samples were incubated with PE Mouse Anti-Human CD11a antibody and FITC Mouse Anti-Human CD18 antibody (BD Pharmingen, San Diego, CA), and analysed using the Flow Nano Analyzer (NanoFCM Inc., Xiamen, China) according to manufacturer's protocol. Briefly, 50 μL of vesicles (10¹⁰ particles/mL) was mixed with 50 μL of antibody for 30 min at 37° C., and then washed with PBS at 100,000×g for 20 min. The labelled vesicles were diluted within the optimal range of particle numbers and analyzed using the NanoFCM software (NanoFCM Profession V1.0).

### Results

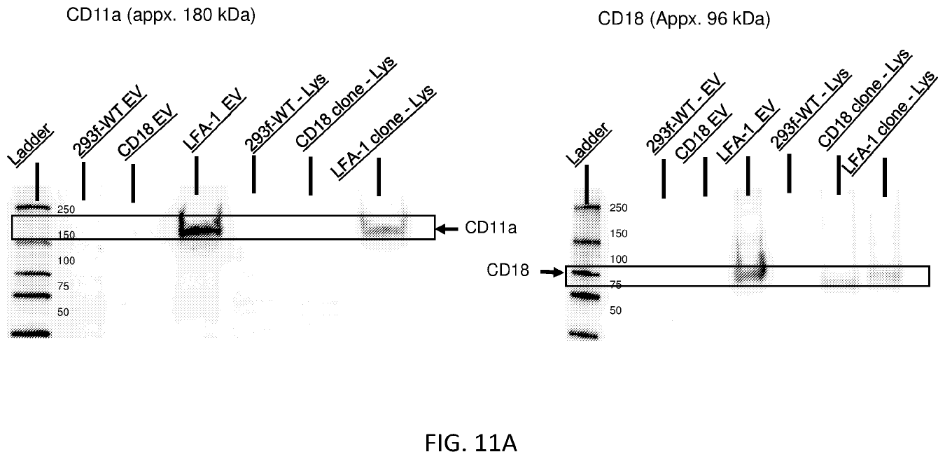

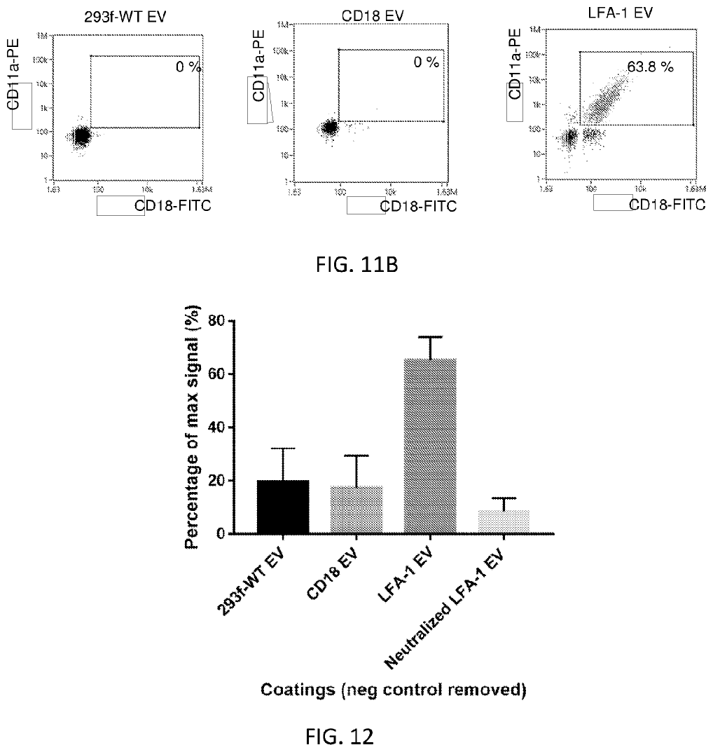

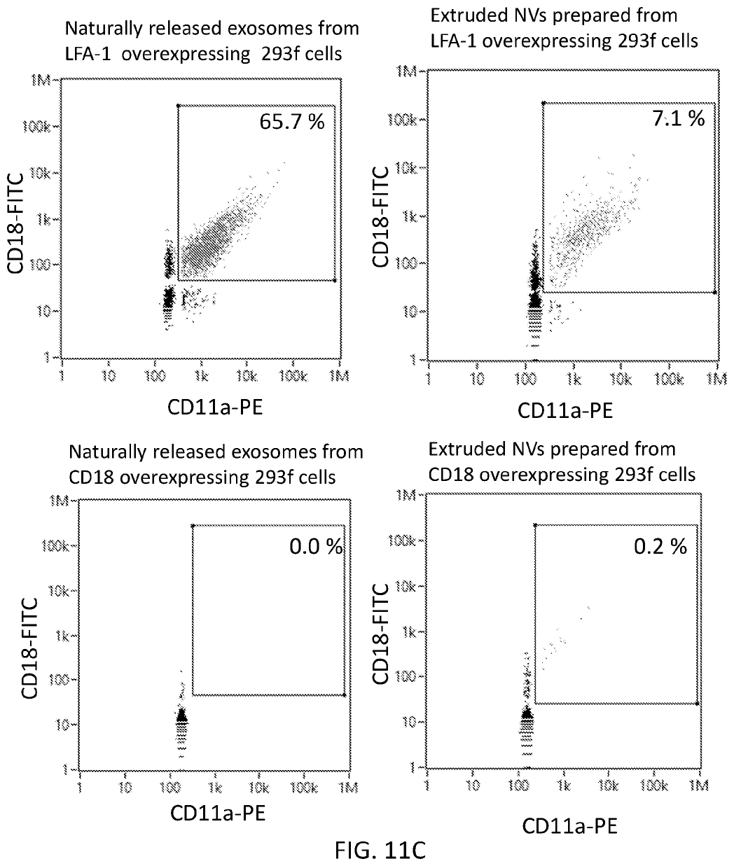

Western blot (FIG. 11A) and Nano-FCM (FIG. 11B) both indicates that the expression of LFA-1 is maintained on the EV isolated from the LFA-1 clone. However, CD18 is not maintained on the EV isolated from the CD18 clone. See also FIG. 11C.

FIG. 11C shows Nano-FCM analysis of exosomes (EVs) isolated from 293f cells genetically modified to overexpress LFA-1, NVs prepared by serial extrusion of 293f cells genetically modified to overexpress LFA-1, EVs isolated from 293f cells genetically modified to overexpress CD18, and NVs prepared by serial extrusion of 293f cells genetically modified to overexpress CD18. As compared to NVs, the exosomes retain a higher level of LFA-1. Thus, in certain cases where cells expressing a receptor for LFA-1 (e.g., ICAM-1) are to be targeted by the vesicles, vesicles made from exosomes would provide for a more efficient delivery as compared to vesicles prepared from extruded vesicles. In other words, the SyEVs would provide for a more efficient delivery as compared to exgNVs.

### Example 5: Confirmation of Functionality of LFA-1 Expressed on EV

### Methods

Reverse binding assay with ICAM-1 expressing cells: EV were diluted in PBS to a concentration 10 μg/mL and incubated overnight in a 96-well plate. The wells in the plate was then blocked using 1% BSA for 1 hour. After the washing step, 50,000 DiO stained cells were added to each well and then incubated for 30 minutes at 37° C. The cells were treated with 15 ng/mL TNF-α to induce higher ICAM-1 expression. The plate was then washed and the fluorescence was analysed with a plate reader.

Uptake experiments: Cells were seeded to a final number of 100,000 cells/well and treated with 15 ng/mL TNF-α 3-4 hours later. The next day 5×10⁹ of the different types of EV were added to each of the respective well and incubated for 30 min at 37° C. Fluorescence data was gathered with a flow cytometer (BD biosciences) and analysed with FlowJo software (Tree Star).

### Results

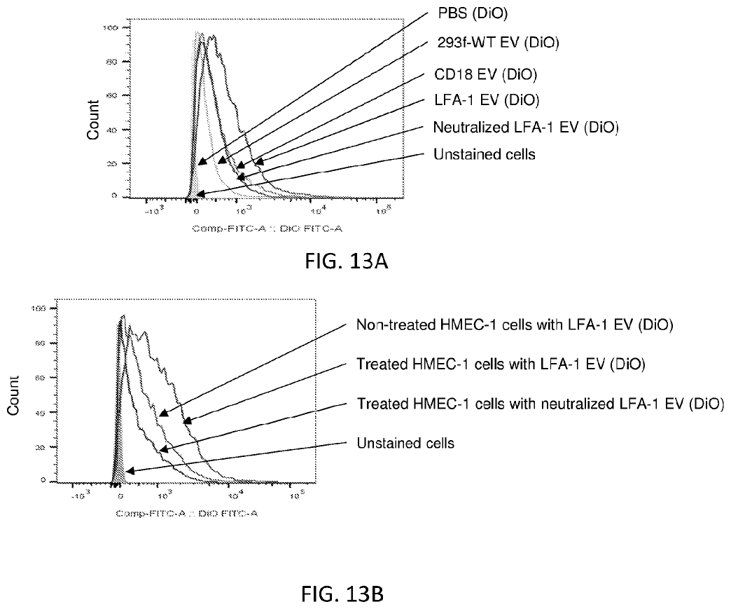

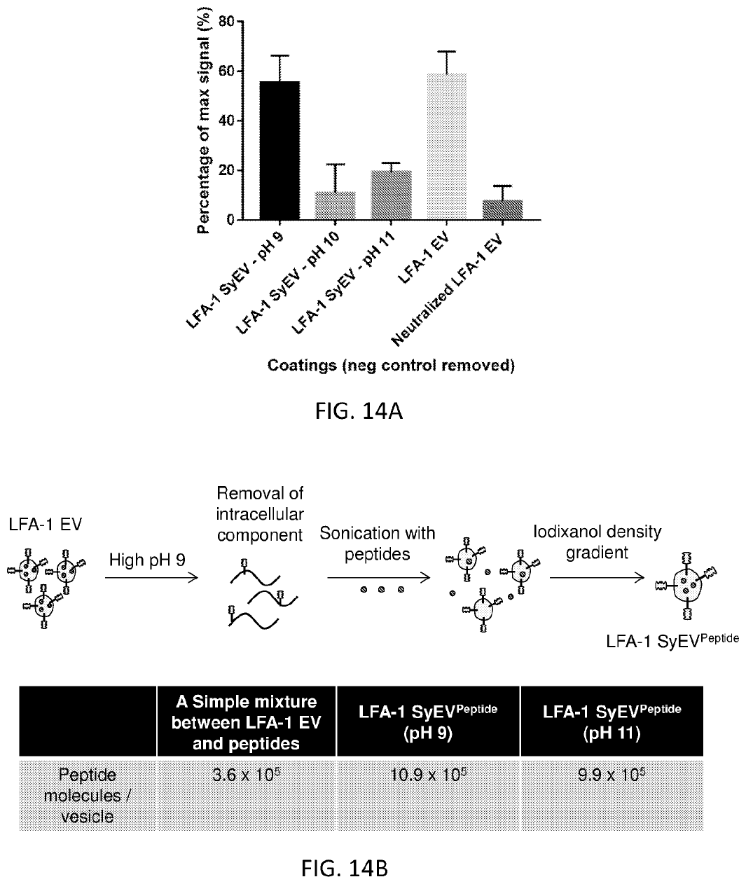

In the reverse binding assays, more cells expressing ICAM-1 were able to bind to the wells with the LFA-1 EV than the wells with other EV and the neutralized LFA-1 EV (FIG. 12). Also, incubating ICAM-1 expressing cells with different types of EV showed that the cells had a higher uptake for the EV with LFA-1 on their surface (FIG. 13A), and ICAM-1-overexpressing cells took up more LFA-1 EV than non-treated cells (FIG. 13B).

### Example 6: The Efficient Delivery of Anti-Inflammatory Peptides Loaded in LFA-1 SyEV

### Methods

Loading LFA-1 SyEV with Myd88 inhibitory peptides: For generating SyEV loaded with anti-Myd88 peptides, the layers formed between 10% and 30% iodixanol after ultracentrifugation at 100,000×g for 2 hours was collected and sonicated with anti-inflammatory peptides for 30 min to generate vesicles loaded with the agent. After sonication, the vesicles were separated from free anti-inflammatory agent by ultracentrifugation (FIG. 14B). The sequence of anti-Myd88 peptide is “RDVLPGT” (SEQ ID NO:2).

Uptake experiments: The peptide-loaded LFA-1 SyEV were diluted in PBS to a concentration 10 μg/mL and incubated overnight in a 96-well plate. The wells in the plate was then blocked using 1% BSA for 1 hour. After the washing step, 50,000 DiO stained cells were added to each well and then incubated for 30 minutes at 37° C. The cells were treated with 15 ng/mL TNF-α to induce higher ICAM-1 expression. The plate was then washed and the fluorescence was analysed with a plate reader.

### Results

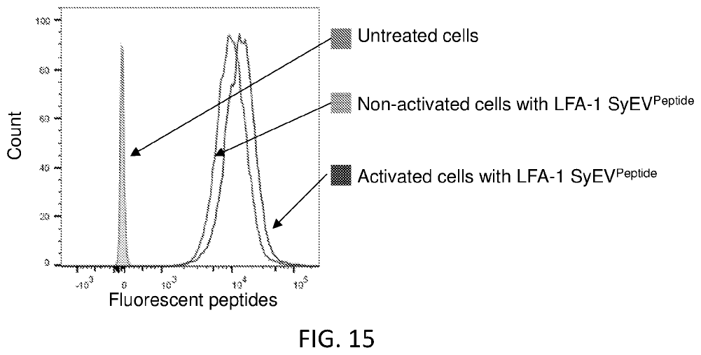

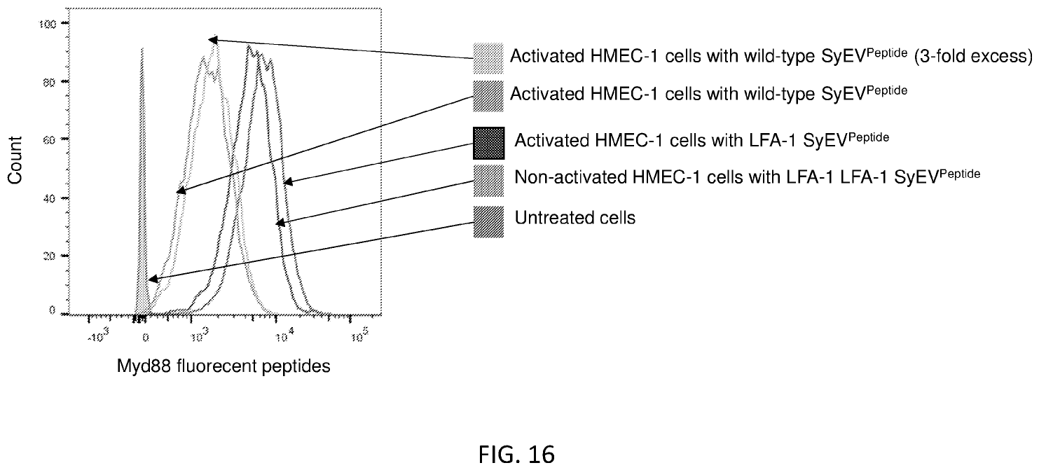

Treating LFA-1 SyEV with different levels of pH showed that the pH 9 treated LFA-1 did not have significant loss in binding efficiency (FIG. 14A), and further testing showed that the pH 9 LFA-1 SyEV had a higher loading capability of Myd88 peptides, compared to high pH non-treated LFA-1 EV (FIG. 14B). Also, we confirmed that the peptides were efficiently delivered to the target cells via SyEV-mediated LFA-1/ICAM-1 interaction (FIG. 15). Moreover, the LFA-1 SyEV were significantly more efficient at delivering peptides to the cells as compared to wild-type SyEV (FIG. 16).

### Example 7: Engineering of EV to Overexpress Mac-1 on their Surface, and Confirmation of the Functionality

### Methods

EV isolation: The cell supernatants were centrifuged at 300×g for 10 min and 2,000×g for 20 min at 4° C. to remove cell debris. The resulting supernatants were ultracentrifuged at 16,500×g for 20 min and 120,000×g for 2.5 h at 4° C. to collect large and small vesicles, respectively. The large and small vesicles were pooled together for further experiment.

Nano-FCM analysis: Different vesicle samples were incubated with PE Mouse Anti-Human CD11 b antibody and FITC Mouse Anti-Human CD18 antibody (BD Pharmingen, San Diego, CA), and analysed using the Flow Nano Analyzer (NanoFCM Inc., Xiamen, China) according to manufacturer's protocol. Briefly, 50 μL of vesicles (10¹⁰ particles/mL) was mixed with 50 μL of antibody for 30 min at 37° C., and then washed with PBS at 100,000×g for 20 min. The labelled vesicles were diluted within the optimal range of particle numbers and analysed using the NanoFCM software (NanoFCM Profession V1.0).

Reverse binding assay with ICAM-1 expressing cells: EV were diluted in PBS to a concentration 10 μg/mL and incubated overnight in a 96-well plate. The wells in the plate were then blocked using 1% BSA for 1 hour. After the washing step, 50,000 DiO stained cells were added to each well and then incubated for 30 minutes at 37° C. The cells were treated with 15 ng/mL TNF-α to induce higher ICAM-1 expression. The plate was then washed, and the fluorescence was analysed with a plate reader.

### Results

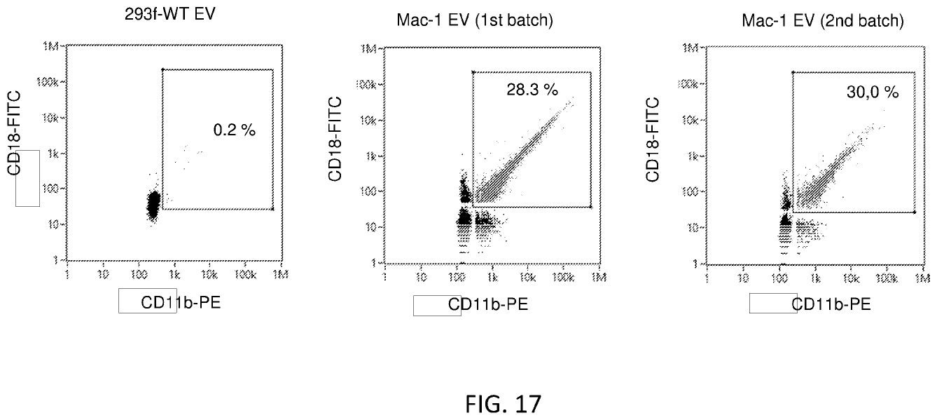

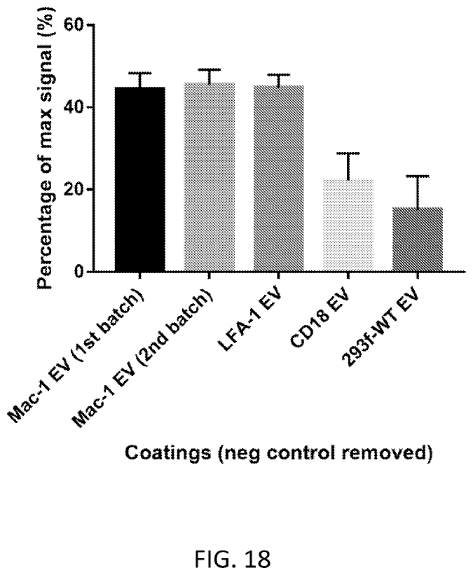

Nano-FCM indicates that the expression of Mac-1 is maintained on the EV isolated from the Mac-1 clone (FIG. 17). In the reverse binding assays, more cells expressing ICAM-1 were able to bind to the wells with the Mac-1 EV, which is similar activity with LFA-1 EV (FIG. 18).

1.-106. (canceled)

107. Vesicles generated from eukaryotic cells and comprising functional integrin on surface of the vesicles and substantially devoid of cytoplasmic content of the eukaryotic cells, wherein the vesicles comprise an anti-inflammatory therapeutic agent.

108. The vesicles of claim 107, wherein the anti-inflammatory agent is cyclosporine A, an inhibitor of Myeloid Differentiation Primary Response 88 (Myd88), an activator of the glucocorticoid receptor, an inhibitor of a non-receptor tyrosine kinase, an inhibitor of Nuclear factor kappa B (NFkB) p50, AKT, extracellular-signal-regulated kinase (ERK), I kappa B kinase gamma (IKKg), Toll Like Receptor 4 (TLR4), Toll Like Receptor 2 (TLR2), Signal transducer and activator of transcription 3 (STAT3), or interleukin-1 receptor-associated kinase 4 (IRAK).

109. The vesicles of claim 108, wherein the inhibitor of MYD88 is a small molecule or a large molecule.

110. The vesicles of claim 109, wherein the small molecule inhibitor of MYD88 is T6167923 or ST2825 and the large molecule inhibitor of MYD88 is a peptide.

111. The vesicles of claim 110, wherein the peptide comprises the amino acid sequence RDVLPGT (SEQ ID NO:2).

112. The vesicles of claim 108, wherein the activator of the glucocorticoid receptor is a small molecule, the inhibitor of a non-receptor tyrosine kinase comprises Nintedanib, Imatinib, Dasatinib, gefitinib, erlotinib, or Lapatinib, the inhibitor of NFkB p50, AKT, ERK, IKKg, TLR4, TLR2, STAT3, or IRAK comprises a peptide.

113. The vesicles of claim 108, wherein the inhibitor of NFkB p50 comprises a peptide comprising the amino acid sequence VQRKRQKLM (SEQ ID NO:3), the inhibitor of AKT comprises a peptide comprising the amino acid sequence AVTDHPDRLWAWEKF (SEQ ID NO:4), the inhibitor of ERK comprises a peptide comprising the amino acid sequence MPKKKPTPIQLNP (SEQ ID NO:5), the inhibitor of IKKg comprises a peptide comprising the amino acid sequence TALDWSWLQTE (SEQ ID NO:6), the inhibitor of TLR4 comprises a peptide comprising the amino acid sequence KYSFKLILAEY (SEQ ID NO:7), the inhibitor of TLR2 comprises a peptide comprising the amino acid sequence PGFLRDPWCKYQML (SEQ ID NO:8), the inhibitor of STAT3 comprises a peptide comprising the amino acid sequence PYLKTKAAVLLPVLLAAP (SEQ ID NO:9), or the inhibitor of IRAK comprises a peptide comprising the amino acid sequence KKARFSRFAGSSPSQSSMVAR (SEQ ID NO:10).

114. The vesicles of claim 107, wherein the vesicles have anti-inflammatory property and enhance the effect of the anti-inflammatory agent.

115. The vesicles of claim 114, wherein the anti-inflammatory property of the vesicles comprises reduction of production and secretion of a proinflammatory cytokine selected from at least one of: IL-2, IL-6, IL-12, or TNF-α.

116. The vesicles of claim 107, wherein the vesicles are generated by a method comprising:
isolating exosomes secreted by the eukaryotic cells;
opening the isolated exosomes by exposing them to an alkaline pH, wherein the mild alkaline pH is sufficient to cause opening of the exosomes while allowing retention of the functional integrin on the surface of the vesicles;
incubating the opened exosomes with a therapeutic agent; and
closing the exosomes by applying energy to the opened exosomes to generate the vesicles.

117. The vesicles of claim 116, wherein the alkaline pH comprises pH7.5-13.5.

118. The vesicles of claim 116, wherein applying energy comprises sonication.

119. The vesicles of claim 107, wherein the vesicles are generated by a method comprising:
disrupting the eukaryotic cells followed by exposure to an alkaline pH prior to forming the vesicles and wherein the vesicles comprise an enrichment of mitochondrial proteins as compared to vesicles made from the same type of cell by exposure to high pH in absence of the disruption step, wherein the disrupting eukaryotic cells comprises sonication or serial extrusion.

120. The vesicles of claim 119, wherein the alkaline pH comprises pH7.5-13.5.

121. The vesicles of claim 107, wherein the eukaryotic cells are human mesenchymal stem cells.

122. The vesicles of claim 107, wherein the eukaryotic cells are HEK 293 cells.

123. A method for treating an inflammatory condition in a subject, the method comprising:
administering a therapeutically effective amount of the vesicles of claim 107 to the subject.

124. A method of making the vesicles of claim 107, the method comprising disrupting the eukaryotic cells followed by exposure to an alkaline pH prior to forming the vesicles and wherein the vesicles comprise an enrichment of mitochondrial proteins as compared to vesicles made from the same type of cell by exposure to high pH in absence of the disruption step, wherein the disrupting the eukaryotic cells comprises sonication or serial extrusion.

125. A method of making the vesicles of claim 107, the method comprising isolating exosomes secreted by the eukaryotic cells;
opening the isolated exosomes by exposing them to an alkaline pH, wherein the mild alkaline pH is sufficient to cause opening of the exosomes while allowing retention of the functional integrin on the surface of the vesicles;
incubating the opened exosomes with the therapeutic agent; and
closing the exosomes by applying energy to the opened exosomes to generate the vesicles.

126. Vesicles produced from a human cell line genetically modified to express lymphocyte function-associated antigen-1 (LFA-1) or macrophage-1 antigen (Mac-1), wherein LFA-1 or Mac-1 is located on surface of the vesicles.


SEQUENCE LISTING (12 sequences)

SEQ ID NO: 1 - protein, 24 aa, Artificial Sequence
  synthetic sequence
DRQIKIWFQNRRMKWKKRDVLPGT

SEQ ID NO: 2 - protein, 7 aa, Artificial Sequence
  synthetic sequence
RDVLPGT

SEQ ID NO: 3 - protein, 9 aa, Artificial Sequence
  synthetic sequence
VQRKRQKLM

SEQ ID NO: 4 - protein, 15 aa, Artificial Sequence
  synthetic sequence
AVTDHPDRLWAWEKF

SEQ ID NO: 5 - protein, 13 aa, Artificial Sequence
  synthetic sequence
MPKKKPTPIQLNP

SEQ ID NO: 6 - protein, 11 aa, Artificial Sequence
  synthetic sequence
TALDWSWLQTE

SEQ ID NO: 7 - protein, 11 aa, Artificial Sequence
  synthetic sequence
KYSFKLILAEY

SEQ ID NO: 8 - protein, 14 aa, Artificial Sequence
  synthetic sequence
PGFLRDPWCKYQML

SEQ ID NO: 9 - protein, 18 aa, Artificial Sequence
  synthetic sequence
PYLKTKAAVLLPVLLAAP

SEQ ID NO: 10 - protein, 21 aa, Artificial Sequence
  synthetic sequence
KKARFSRFAGSSPSQSSMVAR

SEQ ID NO: 11 - RNA, 21 nt, Artificial Sequence
  synthetic sequence
uaaggcuaugaagagauacuu

SEQ ID NO: 12 - RNA, 21 nt, Artificial Sequence
  synthetic sequence
guaucucuucauagccuuauu